(19) United States
(12) Patent Application Publication
(10) Pub. No.: US 2021/0235714 A1
(43) Pub. Date: Aug. 5, 2021

(54) RECOMBINANT MILK PROTEIN POLYMERS

(71) Applicant: PERFECT DAY, INC., BERKELEY, CA (US)

(72) Inventors: Timothy Geistlinger, Oakland, CA (US); Ravirajsinh Jhala, Oakland, CA (US); Bonney Oommen, Salt Lake City, UT (US); Balakrishnan Ramesh, Berkeley, CA (US)

(21) Appl. No.: 17/052,073

(22) PCT Filed: Apr. 30, 2019
(86) PCT No.: PCT/US19/30031

§ 371 Date: Oct. 30, 2020

Related U.S. Application Data
(60) Provisional application No. 62/664,586, filed on Apr. 30, 2018.

(51) Int. Cl. A23C 11/10

(52) U.S. Cl. CPC A23C 11/106

(57) ABSTRACT

Provided are polymers of milk protein monomers, methods of manufacturing such polymers, and compositions comprising such polymers.

### CROSS-REFERENCE TO RELATED APPLICATION

This application claims priority to U.S. Provisional Patent Application No. 62/664,586, filed on Apr. 30, 2018, the entire contents of which are incorporated by reference.

### FIELD OF THE INVENTION

Provided are polymers of milk protein monomers, methods for manufacturing such polymers, and compositions comprising such polymers.

### BACKGROUND OF THE INVENTION

Petroleum-based polymers are used extensively in modern societies, for example, in industrial and consumer products. However, production, use, and disposal of petroleum-based polymers leave a large environmental footprint: the polymers are produced from monomers derived from non-renewable petroleum or natural gas sources, can leach harmful compounds, and are typically disposed of in landfill sites. Disposal via recycling has increased in recent years, but recycling rates remain low. Moreover, recycling of products that comprise different types of petroleum-based polymers and/or processing additives (e.g., fillers, coloring agents, plasticizers) is complex and uneconomical. The problems are not solved by newly developed photodegradable polymers, which are also made from petroleum-derived components, and which typically can be degraded by sunlight only to smaller particles of plastic rather than be decomposed completely.

Biopolymers are derived from renewable materials (e.g., agricultural byproducts), are typically safe, and in many cases are fully biodegradable (i.e., degradable by bacteria, fungi, or other microorganisms). Use of biopolymers for production of consumer goods dates back to ancient times. Traditional starting materials for producing biopolymers include milk proteins, starch, and cellulose.

Milk proteins are categorized as either caseins or whey proteins. Caseins are hydrophobic phosphoproteins with relatively little tertiary structure, and include the αS1-, αS2-, β-, γ-, and κ-casein of bovine milk. Whey proteins are globular proteins, and include the β-lactoglobulin, α-lactalbumin, serum albumin, immunoglobulins, lactoferrin, transferrin, lactoperoxidase, and glycomacropeptides of bovine milk.

Both caseins and whey proteins can form biopolymers with interesting properties. For example, whey protein biopolymers can exhibit excellent adhesive, binding, or coating properties, making them suitable as major ingredients in wood or paper adhesives, binders, protective coatings, plastics, and fabrics. Milk protein polymers have also been used to make ornaments, buttons, buckles, beads, pens, combs, and brushes.

Despite their favorable properties, milk proteins fell from favor as starting materials for biopolymers due to their expensive production via extraction from milk obtained from lactating mature animals. Moreover, such production yields fractions of mixed proteins (e.g., whey or casein protein isolates or protein concentrates comprising mixtures of whey proteins or caseins, respectively, as well as other components of milk (e.g., carbohydrates, minerals, lipids), making studying and predicting of behaviors in polymerization reactions and/or properties of resulting polymers difficult. Lastly, chemically produced monomers can be more easily modified in reaction vessels than milk proteins produced by whole organisms, adding to the versatility of petroleum-based polymers compared to biopolymers.

Therefore, a need exists for methods for producing biopolymers from milk proteins that can compete with production of petroleum-based polymers on the basis of cost and ability to control the behavior of monomers in polymerization, thereby providing greater control over monomer and polymer properties. Furthermore, a need exists for biopolymers of milk proteins produced by such methods that comprise only a limited subset of milk proteins and/or a limited subset of specifically modified milk proteins, and that have specific properties.

### SUMMARY OF THE INVENTION

In one aspect, provided herein is a polymer that comprises a milk protein monomer component and that has a desirable attribute.

In some embodiments, the milk protein monomer component comprised in the polymer provided herein consists of: one or more (e.g., two, three, four, or more) whey protein monomers (e.g., a β-monomer, a α-lactalbumin monomer, a mixture of a β-lactoglobulin monomer and an α-lactalbumin monomer, a mixture of two or more β-lactoglobulin monomers having different post-translational modification (PTMs), a mixture of two or more α-lactalbumin monomers having different PTMs, a mixture of two or more β-lactoglobulin monomers having different PTMs and an α-lactalbumin monomer, a mixture of two or more α-lactalbumin monomers having different PTMs and a β-lactoglobulin monomer, a mixture of two or more β-lactoglobulin monomers having different PTMs and a mixture of two or more α-lactalbumin monomers having different PTMs); one or more (e.g., two, three, four, or more) casein monomers (e.g., a κ-casein monomer, a β-casein monomer, a γ-casein monomer, a mixture of a κ-casein monomer and a β-casein monomer, a mixture of a κ-casein monomer and a γ-casein monomer, a mixture of a β-casein monomer and a γ-casein monomer, a mixture of two or more lc-casein monomers having different PTMs, a mixture of two or more β-casein monomers having different PTMs, a mixture of two or more γ-casein monomers having different PTMs, a mixture of two or more κ-casein monomers having different PTMs and a β-casein monomer, a mixture of two or more κ-casein monomers having different PTMs and a γ-casein monomer, a mixture of two or more β-casein monomers having different PTMs and a κ-casein monomer, a mixture of two or more β-casein monomers having different PTMs and a γ-casein monomer, a mixture of two or more γ-casein monomers having different PTMs and a κ-casein monomer, a mixture of two or more γ-casein monomers having different PTMs and a β-casein monomer, a mixture of two or more κ-casein monomers having different PTMs and/or two or more β-casein monomers having different PTMs and/or two or more γ-casein monomers having different PTMs); or a mixture thereof.

In some embodiments, the milk protein monomer component comprised in the polymer provided herein consists of whey protein monomers and casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises: one or more (e.g., two, three, four, or more) native whey protein monomers (e.g., a native β-lactoglobulin monomer, a native α-lactalbumin monomer, a mixture of a native β-lactoglobulin monomer and a native α-lactalbumin monomer, a mixture of two or more native β-lactoglobulin monomers having different PTMs, a mixture of two or more native α-lactalbumin monomers having different PTMs, a mixture of two or more native β-lactoglobulin monomers having different PTMs and a native α-lactalbumin monomer, a mixture of two or more native α-lactalbumin monomers having different PTMs and a native β-lactoglobulin monomer, a mixture of two or more native β-lactoglobulin monomers having different PTMs and a mixture of two or more native α-lactalbumin monomers having different PTMs); one or more (e.g., two, three, four, or more) native casein monomers (e.g., a native κ-casein monomer, a native β-casein monomer, a native γ-casein monomer, a mixture of a native κ-casein monomer and a native β-casein monomer, a mixture of a native κ-casein monomer and a native γ-casein monomer, a mixture of a native β-casein monomer and a native γ-casein monomer, a mixture of two or more native κ-casein monomers having different PTMs, a mixture of two or more native β-casein monomers having different PTMs, a mixture of two or more native γ-casein monomers having different PTMs, a mixture of two or more native κ-casein monomers having different PTMs and a native β-casein monomer, a mixture of two or more native κ-casein monomers having different PTMs and a native γ-casein monomer, a mixture of two or more native β-casein monomers having different PTMs and a native κ-casein monomer, a mixture of two or more native β-casein monomers having different PTMs and a native γ-casein monomer, a mixture of two or more native γ-casein monomers having different PTMs and a native κ-casein monomer, a mixture of two or more native γ-casein monomers having different PTMs and a native β-casein monomer, a mixture of two or more native κ-casein monomers having different PTMs and/or two or more native β-casein monomers having different PTMs and/or two or more native γ-casein monomers having different PTMs); or a mixture thereof.

In some embodiments, the milk protein monomer component comprised in the polymer provided herein consists of: one or more (e.g., two, three, four, or more) native whey protein monomers (e.g., a native β-lactoglobulin monomer, a native α-lactalbumin monomer, a mixture of a native β-lactoglobulin monomer and a native α-lactalbumin monomer, a mixture of two or more native β-lactoglobulin monomers having different PTMs, a mixture of two or more native α-lactalbumin monomers having different PTMs, a mixture of two or more native β-lactoglobulin monomers having different PTMs and a native α-lactalbumin monomer, a mixture of two or more native α-lactalbumin monomers having different PTMs and a native β-lactoglobulin monomer, a mixture of two or more native β-lactoglobulin monomers having different PTMs and a mixture of two or more native α-lactalbumin monomers having different PTMs); one or more (e.g., two, three, four, or more) native casein monomers (e.g., a native κ-casein monomer, a native β-casein monomer, a native γ-casein monomer, a mixture of a native κ-casein monomer and a native β-casein monomer, a mixture of a native κ-casein monomer and a native γ-casein monomer, a mixture of a native β-casein monomer and a native γ-casein monomer, a mixture of two or more native κ-casein monomers having different PTMs, a mixture of two or more native β-casein monomers having different PTMs, a mixture of two or more native γ-casein monomers having different PTMs, a mixture of two or more native κ-casein monomers having different PTMs and a native β-casein monomer, a mixture of two or more native κ-casein monomers having different PTMs and a native γ-casein monomer, a mixture of two or more native β-casein monomers having different PTMs and a native κ-casein monomer, a mixture of two or more native β-casein monomers having different PTMs and a native γ-casein monomer, a mixture of two or more native γ-casein monomers having different PTMs and a native κ-casein monomer, a mixture of two or more native γ-casein monomers having different PTMs and a native β-casein monomer, a mixture of two or more native κ-casein monomers having different PTMs and/or two or more native β-casein monomers having different PTMs and/or two or more native γ-casein monomers having different PTMs); or a mixture thereof.

In some embodiments, the milk protein monomer component comprised in the polymer provided herein comprises native whey protein monomers and native casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises: one or more (e.g., two, three, four, or more) recombinant whey protein monomers (e.g., a recombinant β-lactoglobulin monomer, a recombinant α-lactalbumin monomer, a mixture of a recombinant β-lactoglobulin monomer and a recombinant α-lactalbumin monomer, a mixture of two or more recombinant β-lactoglobulin monomers having different PTMs, a mixture of two or more recombinant α-lactalbumin monomers having different PTMs, a mixture of two or more recombinant β-lactoglobulin monomers having different PTMs and a recombinant α-lactalbumin monomer, a mixture of two or more recombinant α-lactalbumin monomers having different PTMs and a β-lactoglobulin monomer, a mixture of two or more recombinant β-lactoglobulin monomers having different PTMs and a mixture of two or more recombinant α-lactalbumin monomers having different PTMs); one or more (e.g., two, three, four, or more) recombinant casein monomers (e.g., a recombinant κ-casein monomer, a recombinant β-casein monomer, a recombinant γ-casein monomer, a mixture of a recombinant κ-casein monomer and a recombinant β-casein monomer, a mixture of a recombinant κ-casein monomer and a recombinant γ-casein monomer, a mixture of a recombinant β-casein monomer and a recombinant γ-casein monomer, a mixture of two or more recombinant κ-casein monomers having different PTMs, a mixture of two or more recombinant β-casein monomers having different PTMs, a mixture of two or more recombinant γ-casein monomers having different PTMs, a mixture of two or more recombinant κ-casein monomers having different PTMs and a recombinant β-casein monomer, a mixture of two or more recombinant κ-casein monomers having different PTMs and a γ-casein monomer, a mixture of two or more recombinant β-casein monomers having different PTMs and a recombinant κ-casein monomer, a mixture of two or more recombinant β-casein monomers having different PTMs and a γ-casein monomer, a mixture of two or more recombinant γ-casein monomers having different PTMs and a recombinant κ-casein monomer, a mixture of two or more recombinant γ-casein monomers having different PTMs and a β-casein monomer, a mixture of two or more recombinant κ-casein monomers having different PTMs and/or two or more recombinant β-casein monomers having different PTMs and/or two or more recombinant γ-casein monomers having different PTMs); or a mixture thereof.

In some embodiments, the milk protein monomer component comprised in the polymer provided herein consists of: one or more (e.g., two, three, four, or more) recombinant whey protein monomers (e.g., a recombinant β-lactoglobulin monomer, a recombinant α-lactalbumin monomer, a mixture of a recombinant β-lactoglobulin monomer and a recombinant α-lactalbumin monomer, a mixture of two or more recombinant β-lactoglobulin monomers having different PTMs, a mixture of two or more recombinant α-lactalbumin monomers having different PTMs, a mixture of two or more recombinant β-lactoglobulin monomers having different PTMs and a recombinant α-lactalbumin monomer, a mixture of two or more recombinant α-lactalbumin monomers having different PTMs and a β-lactoglobulin monomer, a mixture of two or more recombinant β-lactoglobulin monomers having different PTMs and a mixture of two or more recombinant α-lactalbumin monomers having different PTMs); one or more (e.g., two, three, four, or more) recombinant casein monomers (e.g., a recombinant κ-casein monomer, a recombinant β-casein monomer, a recombinant γ-casein monomer, a mixture of a recombinant κ-casein monomer and a recombinant β-casein monomer, a mixture of a recombinant κ-casein monomer and a recombinant γ-casein monomer, a mixture of a recombinant β-casein monomer and a recombinant γ-casein monomer, a mixture of two or more recombinant κ-casein monomers having different PTMs, a mixture of two or more recombinant β-casein monomers having different PTMs, a mixture of two or more recombinant γ-casein monomers having different PTMs, a mixture of two or more recombinant κ-casein monomers having different PTMs and a recombinant β-casein monomer, a mixture of two or more recombinant κ-casein monomers having different PTMs and a γ-casein monomer, a mixture of two or more recombinant β-casein monomers having different PTMs and a recombinant κ-casein monomer, a mixture of two or more recombinant β-casein monomers having different PTMs and a γ-casein monomer, a mixture of two or more recombinant γ-casein monomers having different PTMs and a recombinant κ-casein monomer, a mixture of two or more recombinant γ-casein monomers having different PTMs and a β-casein monomer, a mixture of two or more recombinant κ-casein monomers having different PTMs and/or two or more recombinant β-casein monomers having different PTMs and/or two or more recombinant γ-casein monomers having different PTMs); or a mixture thereof.

In some embodiments, the milk protein monomer component comprised in the polymer provided herein comprises recombinant whey protein monomers and recombinant casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises recombinant milk protein monomers and native milk protein monomers at a weight ratio of between 100 to 1 and 1 to 100 (e.g., 100 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10, 1 to 20, 1 to 30, 1 to 40, 1 to 50, 1 to 100).

In some embodiments, the milk protein monomer component comprised in the polymer provided herein comprises recombinant whey protein monomers and native whey protein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises recombinant casein monomers and native casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

The polymer provided herein can further comprise a non-milk protein monomer component derived from any source, including animals, plants, algae, fungi, and microbes. In some embodiments, the polymer provided herein comprises a milk protein monomer component and a non-milk protein monomer component at a weight ratio of between 100 to 1 and 1 to 100 (e.g., 100 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10, 1 to 20, 1 to 30, 1 to 40, 1 to 50, 1 to 100).

In another aspect, provided herein is a method for producing the polymer provided herein. The method comprises the steps for: i) obtaining one or more native and/or recombinant milk protein monomers; ii) polymerizing the one or more native and/or recombinant milk protein monomers to obtain a polymer; and iii) optionally post-processing the polymer.

In another aspect, provided herein is a composition that comprises a polymer provided herein.

### DETAILED DESCRIPTION OF THE INVENTION

Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural, unless otherwise indicated herein or clearly contradicted by context.

The term “and/or” as used herein refers to multiple components in combination or exclusive of one another. For example, “x, y, and/or z” can refer to “x” alone, “y” alone, “z” alone, “x, y, and z”, “(x and y) or z”, or “x or y or z”.

The term “animal protein monomer” as used herein refers to a polypeptide that comprises a sequence of at least 20 amino acids [e.g., at least 20, at least 30, at least 40, at least 50, at least 60, at least 70, at least 80, at least 90, at least 100, or at least 150, and usually not more than 200 amino acids] that is at least 80% identical (e.g., at least 85%, at least 90%, at least 95% identical, at least 99% identical) to a sequence of amino acids in a protein produced by an animal (e.g., any of the animals disclosed herein) and that is repeated in a polymer provided herein.

The term “casein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a casein and that is repeated in a polymer provided herein. Non-limiting examples of caseins include β-casein, γ-casein, κ-casein, α-S1-casein, and α-S-casein.

The term “essentially free of” as used herein refers to the indicated component being either not detectable in the indicated composition by common analytical methods, or to the indicated component being present in such trace amount as to not be functional. The term “functional” as used in this context refers to not contributing to properties of the composition comprising the trace amount of the indicated component, or to not having activity (e.g., chemical activity) in the indicated composition comprising the trace amount of the indicated component, or to not having health-adverse effects upon use or consumption of the composition comprising the trace amount of the indicated component.

The term “filamentous fungal cell” as used herein refers to a cell from any filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungal cell is distinguished from yeast by its hyphal elongation during vegetative growth.

The term “fungal protein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a fungus (e.g., any of the fungi disclosed herein) and that is repeated in a polymer provided herein.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific definition of the fungal kingdom may be adjusted in the future.

The term “host cell” as used herein refers to a cell into which a recombinant polynucleotide has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell but are still included within the scope of the term “host cell” as used herein.

The term “identical” as used herein in the context of polynucleotide or polypeptide sequences refers to the residues in the two sequences that are the same when aligned for maximum correspondence. There are a number of different algorithms known in the art that can be used to measure polynucleotide or polypeptide sequence identity. For instance, sequences can be compared using FASTA (e.g., using its default parameters as provided in the Wisconsin Package Version 10.0, Genetics Computer Group (GCG), Madison, Wis.), Gap (e.g., using its default parameters as provided in the Wisconsin Package Version 10.0, GCG, Madison, Wis.), Bestfit, ClustalW (e.g., using default paramaters of Version 1.83), or BLAST (e.g., using reciprocal BLAST, PSI-BLAST, BLASTP, BLASTN) (see, for example, Pearson. 1990. Methods Enzymol. 183:63; Altschul et al. 1990. J. Mol. Biol. 215:403).

The terms “including,” “includes,” “having,” “has,” “with,” or variants thereof as used herein are intended to be inclusive in a manner similar to the term “comprising”.

The term “microbe” as used herein is an abbreviation for microorganism, and refers to a unicellular organism. As used herein, the term includes all bacteria, archaea, unicellular protista, unicellular animals, unicellular plants, unicellular fungi, unicellular algae, protozoa, and chromista.

The term “milk protein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a protein found in a mammal-produced milk and that is repeated in a polymer provided herein.

The term “milk protein monomer component” as used herein refers to a component that consists of only a subset of whey protein monomers or of only a subset of casein monomers or of a mixture of a subset of whey protein monomers and a subset of casein monomers (i.e., consists of monomers derived from just some but not all proteins present in a whey protein concentrate, whey protein isolate, whey protein hydrolysate, casein concentrate, milk protein concentrate, micellar casein concentrate, sodium caseinate, acid caseinate, or milk protein isolate). The term implies that the milk protein monomers of which the milk protein monomer component consists are the only milk protein monomers comprised in the polymer (i.e., the polymer comprises no other milk protein monomers other than the milk protein monomers of which the milk protein monomer component consists).

The term “native” as used herein refers to what is found in nature.

The term “non-milk protein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a protein not found in a mammal-produced milk and that is repeated in a polymer provided herein.

The term “non-milk protein monomer component” as used herein refers to a component that consists of one or more non-milk protein monomers.

The term “milk protein repeat” as used herein refers to an amino acid sequence that is at least 80% identical (e.g., at least 85%, at least 90%, at least 95% identical, at least 99% identical) to an amino acid sequence in a protein found in a mammal-produced milk (e.g., a whey protein, a casein) and that is present more than once (e.g., at least 2, at least 3, at least 4, at least 5, at least 10, at least 15, at least 20, at least 30, at least 40, at least 50, at least 75, at least 100, at least 150, or at least 200 times) in the recombinant milk protein monomer.

The term “non-native PTM” as used herein refers to a difference in one or more location(s) of one or more PTMs (e.g., glycosylation, phosphorylation) in a protein, and/or a difference in the type of one or more PTMs at one or more location(s) in a protein compared to the native protein (i.e., the protein having “native PTMs”).

The term “non-native reactive site” as used herein refers to a difference in one or more locations and/or types of one or more chemical groups on a polypeptide compared to the native polypeptide (i.e., the polypeptide having “native reactive site”), thereby permitting formation of covalent bonds (i.e., “crosslinks”) of non-native types and/or of non-native numbers and/or with non-native distributions.

The term “non-purified protein” as used herein refers to a protein preparation in which no protein is more concentrated relative to other proteins in the protein preparation than is the case in the natural source from which the protein preparation is derived.

The term “plant protein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a plant (e.g., any of the plants disclosed herein) and that is repeated in a polymer provided herein.

The term “polymeric network” as used herein refers to a network of polymers that are crosslinked with each other via covalent bonds.

The term “polynucleotide” as disclosed herein refers to both sense and antisense strands of RNA, cDNA, genomic DNA, and synthetic forms and mixed polymers of the above. A polynucleotide may be modified chemically or biochemically or may contain non-natural or derivatized nucleotide bases. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are known in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding and other chemical interactions. Such molecules are known in the art and include, for example, molecules in which peptide linkages substitute for phosphate linkages in the backbone of the molecule. Other modifications can include, for example, analogs in which the ribose ring contains a bridging moiety or other structure such as the modifications found in “locked” polynucleotides.

The term “post-translational modification”, or its acronym “PTM”, as used herein refers to the covalent attachment of a chemical group to a protein after protein biosynthesis. PTM can occur on the amino acid side chain of the protein or at its C- or N-termini. Non-limiting examples of PTMs include glycosylation (i.e., covalent attachment to proteins of glycan groups (i.e., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof; see, for example, Deshpande et al. 2008. Glycobiology 18(8):626) via C-linkage, N-linkage, or O-linkage, or via glypiation (i.e., addition of a glycosylphosphatidylinositol anchor) or phosphoglycosylation (i.e., linked through the phosphate of a phospho-serine), phosphorylation (i.e., covalent attachment to proteins of phosphate groups), alkylation (i.e., covalent attachment to proteins of alkane groups (e.g, methane group in methylation), and lipidation (i.e., covalent attachment of a lipid group (e.g., isoprenoid group in prenylation and isoprenylation (e.g., farnesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group), and tyrosine nitration (i.e., attachment to tyrosine residues of proteins of nitrate groups).

The term “purifying” as used herein refers to a protein being substantially separated from chemicals and cellular components (e.g., membrane lipids, chromosomes, proteins). The term does not require (albeit allows) that the protein be separated from all other chemicals and cellular components.

The terms “optional” or “optionally” as used herein refer to a feature or structure being present or not, or an event or circumstance occurring or not. The description includes instances in which a particular feature or structure is present and instances in which the feature or structure is absent, or instances in which the event or circumstance occurs and instances in which the event or circumstance does not occur.

The term “partially purified protein” as used herein refers to a protein preparation in which one or more proteins are between 2-fold and 10-fold more abundant relative to other proteins in the protein preparation than they are present in the natural source from which the protein preparation is derived.

The term “polymer” as used herein refers to a molecule that is composed of repeated protein monomers that are covalently linked, either directly with each other or via intermediary molecules.

The term “protein” as used herein refers to a polypeptide (i.e., polymeric form of amino acids) of any length, which can include polypeptides comprising coded and non-coded amino acids, polypeptides comprising amino acids that occur in nature and those that do not occur in nature, polypeptides comprising chemically or biochemically modified or derivatized amino acids, and polypeptides comprising modified peptide backbones.

The term “protein concentrate” as used herein refers to a protein material that is obtained upon removal of at least a portion (or a substantial portion) of one or more of carbohydrates, lipids, ash, and other minor constituents. It typically comprises between 30% and 80% (e.g., between 30% and 80%, 75%, 70%, 65%, 60%, 55%, 50%, 45%, 40%, or 35%; between 35% and 80%, 75%, 70%, 65%, 60%, 55%, 50%, 45%, or 40%; between 40% and 80%, 75%, 70%, 65%, 60%, 55%, 50%, or 45%; between 45% and 80%, 75%, 70%, 65%, 60%, 55%, or 50%; between 50% and 80%, 75%, 70%, 65%, 60%, or 55%; between 55% and 80%, 75%, 70%, 65%, or 60%; between 60% and 80%, 75%, 70%, or 65%; between 65% and 80%, 75%, or 70%; between 70% and 80%, or 75%; or between 75% and 80%) by weight of protein.

The term “protein isolate” as used herein refers to a protein material that is obtained upon removal of at least a portion (or a substantial portion) of one or more of polysaccharides, soluble carbohydrates, ash, and other minor constituents. It typically comprises at least 80% (i.e., at least 80%, at least 85%, at least 95%, at least 99%) by weight of protein.

The term “purified protein” as used herein refers to a protein preparation in which one or more proteins are at least 10-fold more abundant relative to other proteins present in the protein preparation than they are present in the natural source from which the protein preparation is derived.

The term “purifying” as used herein refers to a protein being substantially separated from cellular components (e.g., cell membrane lipids, chromosomes, other proteins). The term does not require (albeit allows) that the protein be separated from all other chemicals.

The term “reactive site” as used herein refers to a chemical group on a molecule (e.g., polymer) that can form a covalent bond (i.e., “crosslinks”) with a chemical group on another molecule (e.g., another milk protein monomer, a non-milk protein monomer).

The term “recombinant host cell” as used herein refers to a host cell that comprises a recombinant polynucleotide. It should be understood that such term is intended to refer not only to the particular subject cell but also to the progeny of such a cell. Because certain modifications may occur in succeeding generations due to either mutation or environmental influences, such progeny may not be identical to the parent cell but are still included within the scope of the term “recombinant host cell” as used herein.

The term “recombinant milk protein monomer”, “recombinant whey protein monomer”, “recombinant casein monomer”, or “recombinant non-milk protein monomer” as used herein refers to a milk protein monomer, whey protein monomer, casein monomer, or non-milk protein monomer, respectively, that is produced recombinantly (i.e., that is produced by a recombinant host cell).

The term “recombinant polynucleotide” as used herein refers to a polynucleotide that has been removed from its naturally occurring environment, a polynucleotide that is not associated with all or a portion of a polynucleotide abutting or proximal to the polynucleotide when it is found in nature, a polynucleotide that is operatively linked to a polynucleotide that it is not linked to in nature, or a polynucleotide that does not occur in nature. The term can be used, e.g., to describe cloned DNA isolates, or a polynucleotide comprising a chemically synthesized nucleotide analog. A polynucleotide is also considered “recombinant” if it contains any genetic modification that does not naturally occur. For instance, an endogenous polynucleotide is considered a “recombinant polynucleotide” if it contains an insertion, deletion, or substitution of one or more nucleotides that is introduced artificially, e.g., by human intervention. Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome).

The term “vector” as used herein refers to a polynucleotide capable of transporting another polynucleotide to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome.

The term “whey protein monomer” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a whey protein and that is repeated in a polymer provided herein. Non-limiting examples of whey proteins include α-lactalbumin, β-lactoglobulin, lactoferrin, transferrin, serum albumin, lactoperoxidase, and glycomacropeptides.

The term “yeast” as used herein refers to organisms of the order Saccharomycetales, such as Saccharomyces cerevisiae and Pichia pastoris. Vegetative growth of yeast occurs by budding/blebbing of a unicellular thallus, and carbon catabolism may be fermentative.

Recitation of ranges of values herein are merely intended to serve as a shorthand method of referring individually to each separate value inclusively falling within the range, unless otherwise indicated herein, and each separate value is incorporated into the specification as if it were individually recited herein. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

### Polymer Comprising Milk Protein Monomer Component

In one aspect, provided herein is a polymer that comprises a milk protein monomer component and that has a desirable attribute. The polymer provided herein is desirable as it provides advantages in production, use, and disposal, including but not limited to smaller negative impacts on the environment due to the use of renewable natural resources for their production and the potential for biodegradability, as well as petroleum resource independence. Moreover, the polymer provided herein is desirable as it comprises monomers derived from only a subset of milk proteins. Moreover, the polymer provided herein is desirable as it comprises milk protein monomers that via recombinant production can be specifically modified to provide a polymer with a desired attribute.

The invention is based on the discovery that polymers can be produced from only a subset of milk protein monomers. The invention is further based on the discovery that individual milk protein monomers can be produced recombinantly, and that the recombinant milk protein monomers can be used to produce polymers. The invention is further based on the discovery that recombinant milk protein monomers can be engineered to have specific functional properties, and that such engineered recombinant milk protein monomers can be used to produce polymers with desirable attributes.

In some embodiments, the polymer provided herein comprises between 0.1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 100%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 100%, 95%, 90%, 85%, 80%, 75%, 70%, 65%, 60%, 55%, 50%, 45%, 40%, 35%, 30%, 25%, 20%, 15%, 14%, 13%, or 12%; between 12% and 100%, 95%, 90%, 85%, 80%, 75%, 70%, 65%, 60%, 55%, 50%, 45%, 40%, 35%, 30%, 25%, 20%, 15%, 14%, or 13%; between 13% and 100%, 95%, 90%, 85%, 80%, 75%, 70%, 65%, 60%, 55%, 50%, 45%, 40%, 35%, 30%, 25%, 20%, 15%, or 14%; between 14% and 100%, 95%, 90%, 85%, 80%, 75%, 70%, 65%, 60%, 55%, 50%, 45%, 40%, 35%, 30%, 25%, 20%, or 15%; between 15% and 100%, 95%, 90%, 85%, 80%, 75%, 70%, 65%, 60%, 55%, 50%, 45%, 40%, 35%, 30%, 25%, or 20%; between 20% and 100%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 100%, 95%, 90%, 85%, 80%, 75%, 70%, 65%, 60%, 55%, 50%, 45%, 40%, or 35%; between 35% and 100%, 95%, 90%, 85%, 80%, 75%, 70%, 65%, 60%, 55%, 50%, 45%, or 40%; between 40% and 100%, 95%, 90%, 85%, 80%, 75%, 70%, 65%, 60%, 55%, 50%, or 45%; between 45% and 100%, 95%, 90%, 85%, 80%, 75%, 70%, 65%, 60%, 55%, or 50%; between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; or between 85% and 100%, 95%, 90%; between 90% and 100% or 95%, or between 95% and 100% by weight of the milk protein monomer component.

The polymer provided herein can further comprise a non-milk protein monomer component.

In some embodiments, the polymer provided herein comprises between 0.01% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0.05%; between 0.05% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%; between 0.1% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 90%, 85%, 80%, 75%, 70%, 65%, 60%, 55%, 50%, 45%, 40%, 35%, 30%, 25%, 20%, 15%, 14%, 13%, 12%, or 11%; between 11% and 90%, 85%, 80%, 75%, 70%, 65%, 60%, 55%, 50%, 45%, 40%, 35%, 30%, 25%, 20%, 15%, 14%, 13%, or 12%; between 12% and 90%, 85%, 80%, 75%, 70%, 65%, 60%, 55%, 50%, 45%, 40%, 35%, 30%, 25%, 20%, 15%, 14%, or 13%; between 13% and 90%, 85%, 80%, 75%, 70%, 65%, 60%, 55%, 50%, 45%, 40%, 35%, 30%, 25%, 20%, 15%, or 14%; between 14% and 90%, 85%, 80%, 75%, 70%, 65%, 60%, 55%, 50%, 45%, 40%, 35%, 30%, 25%, 20%, or 15%; between 15% and 90%, 85%, 80%, 75%, 70%, 65%, 60%, 55%, 50%, 45%, 40%, 35%, 30%, 25%, or 20%; between 20% and 90%, 85%, 80%, 75%, 70%, 65%, 60%, 55%, 50%, 45%, 40%, 35%, 30%, or 25%; between 25% and 90%, 85%, 80%, 75%, 70%, 65%, 60%, 55%, 50%, 45%, 40%, 35%, or 30%; between 30% and 90%, 85%, 80%, 75%, 70%, 65%, 60%, 55%, 50%, 45%, 40%, or 35%; between 35% and 90%, 85%, 80%, 75%, 70%, 65%, 60%, 55%, 50%, 45%, or 40%; between 40% and 90%, 85%, 80%, 75%, 70%, 65%, 60%, 55%, 50%, or 45%; between 44% and 90%, 85%, 80%, 75%, 70%, 65%, 60%, 55%, or 50%; between 50% and 90%, 85%, 80%, 75%, 70%, 65%, 60%, or 55%; between 55% and 90%, 85%, 80%, 75%, 70%, 65%, or 60%; between 60% and 90%, 85%, 80%, 75%, 70%, or 65%; between 65% and 90%, 85%, 80%, 75%, or 70%; between 70% and 90%, 85%, 80%, or 75%; between 75% and 90%, 85%, or 80%; between 80% and 90%, or 85%; or between 85% and 90% by weight of the non-milk protein monomer component.

In some embodiments, the polymer provided herein comprises a milk protein monomer component and a non-milk protein monomer component at a weight ratio of between 100 to 1 and 1 to 100 (e.g., 100 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10, 1 to 20, 1 to 30, 1 to 40, 1 to 50, 1 to 100).

In some embodiments, the polymer provided herein is essentially free of a component derived from petroleum. In other embodiments, the polymer provided herein comprises 2% or less by weight of a component derived from petroleum.

### Milk Protein Monomer Component

In some embodiments, the milk protein monomer component comprised in the polymer provided herein consists of: one or more (e.g., two, three, four, or more) whey protein monomers (e.g., a β-monomer, a α-lactalbumin monomer, a mixture of a β-lactoglobulin monomer and an α-lactalbumin monomer, a mixture of two or more β-lactoglobulin monomers having different post-translational modification (PTMs), a mixture of two or more α-lactalbumin monomers having different PTMs, a mixture of two or more β-lactoglobulin monomers having different PTMs and an α-lactalbumin monomer, a mixture of two or more α-lactalbumin monomers having different PTMs and a β-lactoglobulin monomer, a mixture of two or more β-lactoglobulin monomers having different PTMs and a mixture of two or more α-lactalbumin monomers having different PTMs); one or more (e.g., two, three, four, or more) casein monomers (e.g., a κ-casein monomer, a β-casein monomer, a κ-casein monomer, a mixture of a κ-casein monomer and a β-casein monomer, a mixture of a κ-casein monomer and a γ-casein monomer, a mixture of a β-casein monomer and a γ-casein monomer, a mixture of two or more κ-casein monomers having different PTMs, a mixture of two or more β-casein monomers having different PTMs, a mixture of two or more γ-casein monomers having different PTMs, a mixture of two or more κ-casein monomers having different PTMs and a β-casein monomer, a mixture of two or more κ-casein monomers having different PTMs and a γ-casein monomer, a mixture of two or more β-casein monomers having different PTMs and a κ-casein monomer, a mixture of two or more β-casein monomers having different PTMs and a γ-casein monomer, a mixture of two or more γ-casein monomers having different PTMs and a κ-casein monomer, a mixture of two or more γ-casein monomers having different PTMs and a β-casein monomer, a mixture of two or more κ-casein monomers having different PTMs and/or two or more β-casein monomers having different PTMs and/or two or more γ-casein monomers having different PTMs); or a mixture thereof.

In some embodiments, the milk protein monomer component comprised in the polymer provided herein consists of whey protein monomers and casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises: one or more (e.g., two, three, four, or more) native whey protein monomers (e.g., a native β-lactoglobulin monomer, a native α-lactalbumin monomer, a mixture of a native β-lactoglobulin monomer and a native α-lactalbumin monomer, a mixture of two or more native β-lactoglobulin monomers having different PTMs, a mixture of two or more native α-lactalbumin monomers having different PTMs, a mixture of two or more native β-lactoglobulin monomers having different PTMs and a native α-lactalbumin monomer, a mixture of two or more native α-lactalbumin monomers having different PTMs and a native β-lactoglobulin monomer, a mixture of two or more native β-lactoglobulin monomers having different PTMs and a mixture of two or more native α-lactalbumin monomers having different PTMs); one or more (e.g., two, three, four, or more) native casein monomers (e.g., a native κ-casein monomer, a native β-casein monomer, a native γ-casein monomer, a mixture of a native κ-casein monomer and a native β-casein monomer, a mixture of a native κ-casein monomer and a native γ-casein monomer, a mixture of a native β-casein monomer and a native γ-casein monomer, a mixture of two or more native κ-casein monomers having different PTMs, a mixture of two or more native β-casein monomers having different PTMs, a mixture of two or more native γ-casein monomers having different PTMs, a mixture of two or more native κ-casein monomers having different PTMs and a native β-casein monomer, a mixture of two or more native κ-casein monomers having different PTMs and a native γ-casein monomer, a mixture of two or more native β-casein monomers having different PTMs and a native κ-casein monomer, a mixture of two or more native β-casein monomers having different PTMs and a native γ-casein monomer, a mixture of two or more native γ-casein monomers having different PTMs and a native κ-casein monomer, a mixture of two or more native γ-casein monomers having different PTMs and a native β-casein monomer, a mixture of two or more native κ-casein monomers having different PTMs and/or two or more native β-casein monomers having different PTMs and/or two or more native γ-casein monomers having different PTMs); or a mixture thereof.

In some embodiments, the milk protein monomer component comprised in the polymer provided herein consists of: one or more (e.g., two, three, four, or more) native whey protein monomers (e.g., a native β-lactoglobulin monomer, a native α-lactalbumin monomer, a mixture of a native β-lactoglobulin monomer and a native α-lactalbumin monomer, a mixture of two or more native β-lactoglobulin monomers having different PTMs, a mixture of two or more native α-lactalbumin monomers having different PTMs, a mixture of two or more native β-lactoglobulin monomers having different PTMs and a native α-lactalbumin monomer, a mixture of two or more native α-lactalbumin monomers having different PTMs and a native β-lactoglobulin monomer, a mixture of two or more native β-lactoglobulin monomers having different PTMs and a mixture of two or more native α-lactalbumin monomers having different PTMs); one or more (e.g., two, three, four, or more) native casein monomers (e.g., a native κ-casein monomer, a native β-casein monomer, a native γ-casein monomer, a mixture of a native κ-casein monomer and a native β-casein monomer, a mixture of a native κ-casein monomer and a native γ-casein monomer, a mixture of a native β-casein monomer and a native γ-casein monomer, a mixture of two or more native κ-casein monomers having different PTMs, a mixture of two or more native β-casein monomers having different PTMs, a mixture of two or more native γ-casein monomers having different PTMs, a mixture of two or more native κ-casein monomers having different PTMs and a native β-casein monomer, a mixture of two or more native κ-casein monomers having different PTMs and a native γ-casein monomer, a mixture of two or more native β-casein monomers having different PTMs and a native κ-casein monomer, a mixture of two or more native β-casein monomers having different PTMs and a native γ-casein monomer, a mixture of two or more native γ-casein monomers having different PTMs and a native κ-casein monomer, a mixture of two or more native γ-casein monomers having different PTMs and a native β-casein monomer, a mixture of two or more native κ-casein monomers having different PTMs and/or two or more native β-casein monomers having different PTMs and/or two or more native γ-casein monomers having different PTMs); or a mixture thereof.

In some embodiments, the milk protein monomer component comprised in the polymer provided herein comprises native whey protein monomers and native casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises: one or more (e.g., two, three, four, or more) recombinant whey protein monomers (e.g., a recombinant β-lactoglobulin monomer, a recombinant α-lactalbumin monomer, a mixture of a recombinant β-lactoglobulin monomer and a recombinant α-lactalbumin monomer, a mixture of two or more recombinant β-lactoglobulin monomers having different PTMs, a mixture of two or more recombinant α-lactalbumin monomers having different PTMs, a mixture of two or more recombinant β-lactoglobulin monomers having different PTMs and a recombinant α-lactalbumin monomer, a mixture of two or more recombinant α-lactalbumin monomers having different PTMs and a β-lactoglobulin monomer, a mixture of two or more recombinant β-lactoglobulin monomers having different PTMs and a mixture of two or more recombinant α-lactalbumin monomers having different PTMs); one or more (e.g., two, three, four, or more) recombinant casein monomers (e.g., a recombinant κ-casein monomer, a recombinant β-casein monomer, a recombinant γ-casein monomer, a mixture of a recombinant κ-casein monomer and a recombinant β-casein monomer, a mixture of a recombinant κ-casein monomer and a recombinant γ-casein monomer, a mixture of a recombinant β-casein monomer and a recombinant γ-casein monomer, a mixture of two or more recombinant κ-casein monomers having different PTMs, a mixture of two or more recombinant β-casein monomers having different PTMs, a mixture of two or more recombinant γ-casein monomers having different PTMs, a mixture of two or more recombinant κ-casein monomers having different PTMs and a recombinant β-casein monomer, a mixture of two or more recombinant κ-casein monomers having different PTMs and a γ-casein monomer, a mixture of two or more recombinant β-casein monomers having different PTMs and a recombinant κ-casein monomer, a mixture of two or more recombinant β-casein monomers having different PTMs and a γ-casein monomer, a mixture of two or more recombinant γ-casein monomers having different PTMs and a recombinant κ-casein monomer, a mixture of two or more recombinant γ-casein monomers having different PTMs and a β-casein monomer, a mixture of two or more recombinant κ-casein monomers having different PTMs and/or two or more recombinant β-casein monomers having different PTMs and/or two or more recombinant γ-casein monomers having different PTMs); or a mixture thereof.

In some embodiments, the milk protein monomer component comprised in the polymer provided herein consists of: one or more (e.g., two, three, four, or more) recombinant whey protein monomers (e.g., a recombinant β-lactoglobulin monomer, a recombinant α-lactalbumin monomer, a mixture of a recombinant β-lactoglobulin monomer and a recombinant α-lactalbumin monomer, a mixture of two or more recombinant β-lactoglobulin monomers having different PTMs, a mixture of two or more recombinant α-lactalbumin monomers having different PTMs, a mixture of two or more recombinant β-lactoglobulin monomers having different PTMs and a recombinant α-lactalbumin monomer, a mixture of two or more recombinant α-lactalbumin monomers having different PTMs and a β-lactoglobulin monomer, a mixture of two or more recombinant β-lactoglobulin monomers having different PTMs and a mixture of two or more recombinant α-lactalbumin monomers having different PTMs); one or more (e.g., two, three, four, or more) recombinant casein monomers (e.g., a recombinant κ-casein monomer, a recombinant β-casein monomer, a recombinant γ-casein monomer, a mixture of a recombinant κ-casein monomer and a recombinant β-casein monomer, a mixture of a recombinant κ-casein monomer and a recombinant γ-casein monomer, a mixture of a recombinant β-casein monomer and a recombinant γ-casein monomer, a mixture of two or more recombinant κ-casein monomers having different PTMs, a mixture of two or more recombinant β-casein monomers having different PTMs, a mixture of two or more recombinant γ-casein monomers having different PTMs, a mixture of two or more recombinant κ-casein monomers having different PTMs and a recombinant β-casein monomer, a mixture of two or more recombinant κ-casein monomers having different PTMs and a γ-casein monomer, a mixture of two or more recombinant β-casein monomers having different PTMs and a recombinant κ-casein monomer, a mixture of two or more recombinant β-casein monomers having different PTMs and a γ-casein monomer, a mixture of two or more recombinant γ-casein monomers having different PTMs and a recombinant κ-casein monomer, a mixture of two or more recombinant γ-casein monomers having different PTMs and a β-casein monomer, a mixture of two or more recombinant κ-casein monomers having different PTMs and/or two or more recombinant β-casein monomers having different PTMs and/or two or more recombinant γ-casein monomers having different PTMs); or a mixture thereof.

In some embodiments, the milk protein monomer component comprised in the polymer provided herein comprises recombinant whey protein monomers and recombinant casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises recombinant milk protein monomers and native milk protein monomers at a weight ratio of between 100 to 1 and 1 to 100 (e.g., 100 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10, 1 to 20, 1 to 30, 1 to 40, 1 to 50, 1 to 100).

In some embodiments, the milk protein monomer component comprised in the polymer provided herein comprises recombinant whey protein monomers and native whey protein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

In some embodiments, the milk protein monomer component comprised in the polymer provided herein comprises recombinant casein monomers and native casein monomers at a weight ratio of between 10 to 1 and 1 to 10 (e.g., 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10).

The recombinant or native milk protein monomers comprised in the milk protein component of the polymer provided herein can be derived from any mammalian species, including but not limited to cow, human, sheep, goat, buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

Non-limiting examples of polynucleotide sequences encoding whey proteins and caseins are disclosed in PCT filing PCT/US2015/046428 filed Aug. 21, 2015, and PCT filing PCT/US2017/48730 filed Aug. 25, 2017, which are hereby incorporated herein in their entireties.

In embodiments in which the milk protein component consists of or comprises a recombinant milk protein monomer, the recombinant milk protein monomer can lack an epitope that can elicit an immune response in a human or animal. Such recombinant milk protein monomer is particularly suitable for use in a polymer, and a composition comprising such polymer, that is edible or ingested (e.g., food product, pharmaceutical formulation, hemostatic product).

In embodiments in which the milk protein component consists of or comprises a recombinant milk protein monomer having a PTM, the PTM can be a native PTM, a non-native PTM, or a mixture thereof. In some embodiments, the type and/or number of PTMs of the recombinant milk protein monomer confers a desirable attribute (e.g., viscosity, elastomeric properties, tensile strength, hardness, stiffness, stability, crystallinity, transparency, translucence, opaqueness, chemical resistance) on the polymer provided herein.

In embodiments in which the milk protein component consists of or comprises a recombinant milk protein monomer having a reactive site, the reactive site can be a native reactive site, a non-native reactive site, or a mixture of at least one native reactive site and at least one non-native reactive site. Non-limiting examples of reactive sites include sulfhydryls, primary amines, carboxyls, carboxamides, and hydroxyls (see, for example, (Means & Feeney. 1971. Chemical Modification of Proteins. Holden Day, Inc. San Francisco, Cambridge, London, Amsterdam). In some embodiments, the type and/or number of reactive sites of the milk protein monomer confers a desirable attribute (e.g., viscosity, elastomeric properties, tensile strength, hardness, stiffness, stability, crystallinity, transparency, translucence, opaqueness, chemical resistance) on the polymer provided herein. For example, a smaller number of reactive sites can reduce the viscosities and/or elastomeric properties and/or tensile strengths and/or hardness and/or water resistance of the polymers; whereas a larger number of reactive sites can increase the viscosities and/or elastomeric properties and/or tensile strengths and/or hardness and/or water resistance of the polymers. Methods for introducing or removing reactive sites in proteins are known in the art (see, for example, Means, G A & Feeney, R E. 1971. Chemical Modification of Proteins. Holden Day, Inc. San Francisco, Cambridge, London, Amsterdam), and include deleting or inserting amino acid residues comprising reactive sites (e.g., cysteine residues, lysine residues, glutamic acid residues, aspartic acid residues, non-natural amino acids (e.g., selenocysteine, methylated amino acids, argenylated amino acids, acylated amino acids, biotinylated amino acids).

In embodiments in which the milk protein component consists of or comprises a recombinant milk protein monomer, the recombinant milk protein monomer can have a milk protein repeat. A milk protein repeat may comprise at least 10, at least 20, at least 30, at least 40, at least 50, at least 75, at least 100, or at least 150, and usually not more than 200 amino acids. A milk protein repeat in a recombinant milk protein monomer can be consecutive (i.e., have no intervening amino acid sequences) or non-consecutive (i.e., have intervening amino acid sequences). When present non-consecutively, the intervening amino acid sequence may play a passive role in providing molecular weight without introducing undesirable properties, or may play an active role in providing for particular properties (e.g., solubility, biodegradability, binding to other molecules).

### Non-Milk Protein Monomer Component

The optional non-milk protein monomer component comprised in the polymer provided herein can comprise non-milk protein monomers derived from any source. Non-limiting examples of such sources include animals, plants, algae, fungi, and microbes.

Non-limiting examples of animals include insects (e.g., fly), mammals (e.g. cow, sheep, goat, rabbit, pig, human), and birds (e.g., chicken).

Non-limiting examples of plants include cycads, ginkgo biloba, conifers, cypress, junipers, thuja, cedarwood, pines, angelica, caraway, coriander, cumin, fennel, parsley, dill, dandelion, helichrysum, marigold, mugwort, safflower, camomile, lettuce, wormwood, calendula, citronella, sages, thyme, chia seed, mustard, olive, coffee, capsicum, eggplant, paprika, cranberry, kiwi, vegetable plants (e.g., carrot, celery), tagetes, tansy, tarragon, sunflower, wintergreen, basil, hyssop, lavender, lemon verbena, marjoram, melissa, patchouli, pennyroyal, peppermint, rosemary, sesame, spearmint, primroses, samara, pepper, pimento, potato, sweet potato, tomato, blueberry, nightshades, petunia, morning glory, lilac, jasmin, honeysuckle, snapdragon, psyllium, wormseed, buckwheat, amaranth, chard, quinoa, spinach, rhubarb, jojoba, cypselea, chlorella, marula, hazelnut, canola, kale, bok choy, rutabaga, frankincense, myrrh, elemi, hemp, pumpkin, squash, curcurbit, manioc, dalbergia, legume plants (e.g., alfalfa, lentils, beans, clovers, peas, fava coceira, frijole bola roja, frijole negro, lespedeza, licorice, lupin, mesquite, carob, soybean, peanut, tamarind, wisteria, cassia, chickpea/garbanzo, fenugreek, green pea, yellow pea, snow pea, lima bean, fava bean), geranium, flax, pomegranate, cotton, okra, neem, fig, mulberry, clove, eucalyptus, tea tree, niaouli, fruiting plants (e.g., apple, apricot, peach, plum, pear, nectarine), strawberry, blackberry, raspberry, cherry, prune, rose, tangerine, citrus (e.g., grapefruit, lemon, lime, orange, bitter orange, mandarin), mango, citrus bergamot, buchu, grape, broccoli, brussels, sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, curcuma aromatica, ginger, lemon grass, oat, palm, pineapple, rice, rye, sorghum, triticale, turmeric, yam, bamboo, barley, cajuput, canna, cardamom, maize, oat, wheat, cinnamon, sassafras, lindera benzoin, bay laurel, avocado, ylang-ylang, mace, nutmeg, moringa, horsetail, oregano, cilantro, chervil, chive, aggregate fruits, grain plants, herbal plants, leafy vegetables, non-grain legume plants, nut plants, succulent plants, land plants, water plants, delbergia, millets, drupes, schizocarps, flowering plants, non-flowering plants, cultured plants, wild plants, trees, shrubs, flowers, grasses, herbaceous plants, brushes, lianas, cacti, tropical plants, subtropical plants, temperate plants, and derivatives and crosses thereof.

Non-limiting examples of algae include green algae (e.g., Chlorella vulgaris, Chlorealla pyrenoidosa), brown algae (e.g., Alaria marginata, Analipus japonicus, Ascophyllum nodosum, Ecklonia sp, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome), red algae (e.g., Chondrus crispus, Chondrus ocellatus, Eucheuma cottonii, Eucheuma spinosum, Furcellaria fastigiata, Gracilaria bursa-pastoris, Gracilaria lichenoides, Gloiopeltis furcata, Gigartina acicularis, Gigartina bursa-pastoris, Gigartina pistillata, Gigartina radula, Gigartina skottsbergii, Gigartina stellata, Palmaria palmata, Porphyra columbina, Porphyra crispata, Porhyra deutata, Porhyra perforata, Porhyra suborbiculata, Porphyra tenera, Porphyridium cruentum, Porphyridium purpureum, Porphyridium aerugineum, Rhodella maculate, Rhodella reticulata, Rhodella violacea, Rhodymenia palmata), and derivatives and crosses thereof.

Non-limiting examples of fungi include Aspergillus nidulans, Aspergillus niger, Aspergillus niger var. awamori, Aspergillus oryzae, Candida albicans, Candida etchellsii, Candida guilliermondii, Candida humilis, Candida lipolytica, Candida pseudotropicalis, Candida utilis, Candida versatilis, Chrysosporium lucknowense, Debaryomyces hansenii, Endothia parasitica, Eremothecium ashbyii, Fusarium gramineum, Fusarium moniliforme, Fusarium venenatum, Hansenula polymorpha, Kluyveromyces lactis, Kluyveromyces marxianus, Kluyveromyces marxianus var. lactis, Kluyveromyces thermotolerans, Morteirella vinaceae var. raffinoseutilizer, Mucor miehei, Mucor miehei var. Cooney et Emerson, Mucor pusillus Lindt, Myceliophthora thermophile, Neurospora crassa, Penicillium roquefortii, Physcomitrella patens, Pichia pastoris, Pichia finlandica, Pichia trehalophila, Pichia koclamae, Pichia membranaefaciens, Pichia minuta, Ogataea minuta, Pichia lindneri, Pichia opuntiae, Pichia thermotolerans, Pichia salictaria, Pichia guercuum, Pichia pijperi, Pichia stiptis, Pichia methanolica, Rhizopus niveus, Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum, Sporidiobolus johnsonii, Sporidiobolus salmonicolor, Sporobolomyces roseus, Trichoderma reesei, Xanthophyllomyces dendrorhous, Yarrowia lipolytica, Zygosaccharomyces rouxii, and derivatives and crosses thereof.

Non-limiting examples of microbes include firmicutes, cyanobacteria (blue-green algae), oscillatoriophcideae, bacillales, lactobacillales, oscillatoriales, bacillaceae, lactobacillaceae, Acetobacter suboxydans, Acetobacter xylinum, Actinoplane missouriensis, Arthrospira platensis, Arthrospira maxima, Bacillus cereus, Bacillus coagulans, Bacillus subtilus, Bacillus cerus, Bacillus licheniformis, Bacillus stearothermophilus, Bacillus subtilis, Escherichia coli, Lactobacillus acidophilus, Lactobacillus bulgaricus, Lactococcus lactis, Lactococcus lactis Lancefield Group N, Lactobacillus reuteri, Leuconostoc citrovorum, Leuconostoc dextranicum, Leuconostoc mesenteroides, Micrococcus lysodeikticus, Spirulina, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, Tetrahymena thermophile, Tetrahymena hegewischi, Tetrahymena hyperangularis, Tetrahymena malaccensis, Tetrahymena pigmentosa, Tetrahymena pyriformis, Tetrahymena vorax, Xanthomonas campestris, and derivatives and crosses thereof.

In some embodiments, the non-milk protein monomer is an animal protein monomer. Non-limiting examples of such proteins include collagen, albumin, and ovalalbumin.

In some embodiments, the non-milk protein monomer is a plant protein monomer. Non-limiting examples of such proteins include pea proteins and potato proteins.

In some embodiments, the non-milk protein monomer is a fungal protein monomer. Non-limiting examples of such proteins include glucoamylase, xylanase, amylase, glucanase, member of the SUN family (Sim1p, Uth1p, Nca3p, Sun4p), elongation factor 1-alpha, mitochondrial leucyl-tRNA synthetase, alpha-amylase, alpha-galactosidase, cellulase, endo-1,4-beta-xylanase, endoglucanase, exo-1,4-beta-xylosidase, glucoamylase, peptidase, aspergillopepsin-1, 1,4-beta-D-glucan cellobiohydrolase A, alpha-galactosidase A, alpha-galactosidase B, alpha-galactosidase D, alpha-glucuronidase A, beta-galactosidase C, glucan 1,3-betα-glucosidase A, hydrophobin, and glucan endo-1,3-beta-glucosidase eglC.

In some embodiments, the non-milk protein monomer is derived from a non-milk protein that is secreted by a host cell (e.g., any of the animal, plant, algae, fungal, or microbial native or recombinant host cells disclosed herein). Suitable secreted non-milk protein monomers can be identified, for example, by obtaining a secretome (i.e., secreted proteins, obtained by, for example, culturing animal, plant, algae, fungal, or microbial cells in liquid culture, removing the cells from the cell culture (e.g., via centrifugation), and optionally concentrating the remaining culture medium; or by sequencing genomes and in silico identifying secreted proteins (see, for example, Mattanovich et al. 2009. Microbial Cell Factories 8:29), whole cell extracts, or fractionated whole cell extracts; optionally partially digesting, glycosylating, phosphorylating, or otherwise enzymatically treating the proteins; and then screening them in assays (e.g., high-throughput assays), e.g., for proteins that have similar, identical, or different properties compared to milk proteins). Suitable secreted non-milk protein monomers can also be identified by screening calcium-enriched fractions of soy proteins, e.g., for proteins that have similar, identical, or different properties compared to milk protein monomers. Non-limiting examples of suitable secreted non-milk protein monomers are provided in PCT filing PCT/US2017/48730 filed Aug. 25, 2017. In some embodiments, the non-milk protein monomer is a secreted fungal protein monomer (i.e., a protein that is secreted by a fungus [e.g., any of the fungi disclosed herein]).

In some embodiments, the non-milk protein component comprises a recombinant non-milk protein monomer. In some such embodiments, the recombinant non-milk protein monomer has a non-native PTM and/or lacks an epitope that can elicit an immune response in a human or animal.

### Attributes

The polymer provided herein has a desirable attribute. In some embodiments, the desirable attribute is an attribute of a petroleum-based polymer.

In some embodiments, the desirable attribute is a desirable crystallinity A desirable crystallinity can be a crystallinity that provides for a specific translucence, opaqueness, or transparency. For example, a higher crystallinity generally allows less light to pass through a polymer, affecting translucence or opaqueness of the polymer, as well as mechanical strength, stiffness, chemical resistance, and stability. Methods for measuring crystallinity are known in the art, and include differential scanning calorimetry (DSC) (B. Wunderlich, Thermal Analysis, Academic Press, 1990, pp. 417-431; TN 48, “Polymer Heats of Fusion”, TA Instruments, New Castle, Del.).

In some embodiments, the desirable attribute is a desirable viscosity and/or density. Methods for measuring viscosity and density are known in the art, and include viscometric methods, rotational viscometric methods, capillary viscometric methods, vibratory viscometric methods, ultrasonic pulse echo methods, pycnometric methods, densymetric methods, and areometric methods (see, for example, Kazys & Rekuviene 2011 Ultragarsas (Ultrasound) 66(4):20-25).

In some embodiments, the desirable attribute is a desirable biodegradability. Methods for measuring biodegradability are known in the art, and include OECD 306 Biodegradability in Sea Water, OECD 311 (ASTM E2170) Anaerobic Biodegradability/Biochemical Methane Potential, ASTM D5338 Aerobic biodegradability/composting assay, ASTM D5511 Anaerobic biodegradability/“landfill simulation”, ASTM D5988 (ISO17556) Biodegradation in soil.

In various other embodiments, the desirable attribute is selected from a desirable color, shape (e.g., length, width, uniformity), shape retention, adhesiveness, reaction to moisture, allergenicity, charge, hydrophobicity, hydrophilicity, texture, thickness, smoothness, hardness, tensile strength, digestibility, solvation, chemical reactivity, permeability, melting temperature, brittleness, toughness, creep or cold flow, porosity, swelling, barrier resistance, impact resistance, gas permeability, electrical conductivity, thermal conductivity, elastic modulus, flexibility, susceptibility to thermal degradation, film-forming properties, adhesiveness, surface active properties, water resistance, mechanical properties, stability of interface, surface tension, release of associated/bound compounds, gas content, strength-at-break, glass transition temperature, and shaping temperature. Methods for measuring such attributes are known in the art, and include but are not limited to sheer stress analysis, storage modulus analysis, texture profile analysis using a texture analyzer, dynamic rheology, viscosity analysis, flow analysis, melt analysis, spectral analysis (e.g., using a spectrophotometer or colorimeter (e.g., a Microcolor tristimulus colorimeter (Dr. Bruno Lange GmbH, Berlin, Germany) and the L*a*b color space according to CIE-LAB (Commission Internationale de l'Éclairage, 1971), and assessments by expert human subjects.

### Supplemented Polymers

In a further aspect, the present invention provides a supplemented polymer that is produced by supplementing a petroleum-derived polymer (i.e., a polymer that comprises one or more petroleum-derived monomers) with one or more recombinant milk protein monomers.

The amount of the one or more of the recombinant milk protein monomers in relation to the amount of the petroleum-derived monomers during blending can vary. In some embodiments, the supplemented polymers comprise between 1% and 99%, 95%, 90%, 80%, 70%, 60%, 50%, 40%, 30%, 20%, 10%, 7%, 5%, 3%, or 2%; between 2% and 99%, 95%, 90%, 80%, 70%, 60%, 50%, 40%, 30%, 20%, 10%, 7%, 5%, or 3%; between 3% and 99%, 95%, 90%, 80%, 70%, 60%, 50%, 40%, 30%, 20%, 10%, 7%, or 5%; between 5% and 99%, 95%, 90%, 80%, 70%, 60%, 50%, 40%, 30%, 20%, 10%, or 7%; between 7% and 99%, 95%, 90%, 80%, 70%, 60%, 50%, 40%, 30%, 20%, or 10%; between 10% and 99%, 95%, 90%, 80%, 70%, 60%, 50%, 40%, 30%, or 20%; between 20% and 99%, 95%, 90%, 80%, 70%, 60%, 50%, 40%, or 30%; between 30% and 99%, 95%, 90%, 80%, 70%, 60%, 50%, or 40%; between 40% and 99%, 95%, 90%, 80%, 70%, 60%, or 50%; between 50% and 99%, 95%, 90%, 80%, 70%, or 60%; between 60% and 99%, 95%, 90%, 80%, or 70%; between 70% and 99%, 95%, 90%, or 80%; between 80% and 99%, 95%, or 90%; between 90% and 99%, or 95%; or between 95% and 99% by weight of the one or more petroleum-derived monomers; and between 1% and 99%, 95%, 90%, 80%, 70%, 60%, 50%, 40%, 30%, 20%, 10%, 7%, 5%, 3%, or 2%; between 2% and 99%, 95%, 90%, 80%, 70%, 60%, 50%, 40%, 30%, 20%, 10%, 7%, 5%, or 3%; between 3% and 99%, 95%, 90%, 80%, 70%, 60%, 50%, 40%, 30%, 20%, 10%, 7%, or 5%; between 5% and 99%, 95%, 90%, 80%, 70%, 60%, 50%, 40%, 30%, 20%, 10%, or 7%; between 7% and 99%, 95%, 90%, 80%, 70%, 60%, 50%, 40%, 30%, 20%, or 10%; between 10% and 99%, 95%, 90%, 80%, 70%, 60%, 50%, 40%, 30%, or 20%; between 20% and 99%, 95%, 90%, 80%, 70%, 60%, 50%, 40%, or 30%; between 30% and 99%, 95%, 90%, 80%, 70%, 60%, 50%, or 40%; between 40% and 99%, 95%, 90%, 80%, 70%, 60%, or 50%; between 50% and 99%, 95%, 90%, 80%, 70%, or 60%; between 60% and 99%, 95%, 90%, 80%, or 70%; between 70% and 99%, 95%, 90%, or 80%; between 80% and 99%, 95%, or 90%; between 90% and 99%, or 95%; or between 95% and 99% by weight of the ore or more of recombinant milk protein monomers.

### Method for Producing a Polymer of Recombinant Milk Protein Monomers

In another aspect, provided herein is a method for producing the polymer provided herein. In some embodiments, the present invention provides a method comprising steps for: i) obtaining one or more native and/or recombinant milk protein monomers; ii) polymerizing the one or more native and/or recombinant milk protein monomers to obtain a polymer; and iii) optionally post-processing the polymer.

### Obtaining Native and/or Recombinant Milk Protein Monomer

A recombinant milk protein monomer can be obtained by culturing a suitable recombinant host cell comprising a recombinant polynucleotide (e.g., recombinant vector) encoding the recombinant milk protein monomer in culture media under conditions suitable for production and/or secretion of the recombinant milk protein monomer. The method can further comprise the steps of: a) obtaining a recombinant polynucleotide encoding the milk protein monomer, and introducing the recombinant polynucleotide into host cell to obtain the recombinant host cell; and/or b) isolating (i.e., purifying) the recombinant milk protein monomer; and/or c) post-processing the recombinant milk protein monomer.

The recombinant polynucleotide can be prepared by any suitable method known in the art, including, without limitation, direct chemical synthesis and cloning. The recombinant polynucleotide typically comprises one or more expression cassettes, wherein each expression cassette comprises: a promoter (e.g., a fungal promoter), an optional signal sequence (i.e., a sequence that encodes a peptide that mediates the delivery of a nascent protein attached to the peptide to the exterior of the cell in which the nascent protein is synthesized), a sequence encoding the milk protein monomer, and a termination sequence (or multiple termination sequences), wherein the promoter is operably linked in sense orientation to the optional signal sequence (i.e., the promoter and optional signal sequence and subsequent sequence encoding the milk protein monomer are positioned such that the promoter is effective for regulating transcription of the optional signal sequence and sequence encoding the milk protein monomer), the optional signal sequence is operably linked in sense orientation to the sequence encoding the milk protein monomer (i.e., the signal sequence and sequence encoding the milk protein monomer are positioned such that transcription and translation produces a recombinant milk protein monomer comprising a functional signal sequence), and the termination sequence is operably linked to the sequence encoding the milk protein monomer (i.e., the sequence encoding the milk protein monomer and the termination sequence are positioned such that the terminator is effective for terminating transcription of the optional signal sequence and sequence encoding the milk protein monomer).

The promoter may be any suitable promoter that is functional in the recombinant host cell. In some embodiments, the promoter is a constitutive promoter. In other embodiments, the promoter is an inducible promoter or a repressible promoter (e.g., a promoter that is induced or repressed in the presence of glucose, galactose, lactose, sucrose, cellulose, sophorose, gentiobiose, sorbose, disaccharides that induce the cellulase promoters, starch, tryptophan, or phosphate).

Non-limiting examples of suitable promoters for use in recombinant fungal host cells include promoters, and functional parts thereof, of genes encoding any of the following proteins: glucoamylase (e.g., glaA of Aspergillus niger, Aspergillus awamori, Aspergillus japonicus, Aspergillus tubingensis, Aspergillus foetidus, or Aspegillus carbonarius), amylase (e.g., Aspergillus oryzae TAKA amylase, Aspergillus niger neutral alpha-amylase, Aspergillus niger acid stable alpha-amylase, fungal α-amylase [amy], bacterial alpha-amylase), protease (e.g., Rhizomucor miehei aspartic protease, Aspergillus oryzae alkaline protease, Fusarium oxysporum trypsin-like protease, Trichoderma reesei protease), lipase (e.g., Rhizomucor miehei lipase), isomerase (e.g., Aspergillus oryzae triose phosphate isomerase, fungal triose phosphate isomerase [tpi], yeast triosephosphate isomerase), acetamidase (e.g., Aspergillus nidulans or Aspergillus oryzae or other fungal acetamidase [amdS]), dehydrogenase (e.g., fungal alcohol dehydrogenase [adhA], fungal glyceraldehyde-3-phosphate dehydrogenase [gpd], yeast alcohol dehydrogenase), xylanase (e.g., fungal xylanase [xlnA], Trichoderma xylanases [xyn1, xyn2, bxl1]), kinase (e.g., yeast 3-phosphoglycerate kinase), hydrolase (e.g., fungal cellobiohydrolase I [cbh1], Trichoderma hydrolases [cbh2, egl1, egl2]), phosphatase (e.g., Fusarium acid phosphatase), and other fungal proteins (e.g., fungal endo α-L-arabinase [abnA], fungal α-L-arabinofuranosidase A [abfA], fungal α-L-arabinofuranosidase B [abfB], fungal phytase, fungal ATP-synthetase, fungal subunit 9 [oliC], fungal sporulation-specific protein [Spo2], fungal SSO, yeast alcohol oxidase, yeast lactase, Neurospora crassa CPC1, Aspergillus nidulans trpC, fungal chitinolytic enzymes [e.g., endo- & exo-chitinase, beta-glucanase], fungal VAMP-associated proteins [VAPs], fungal translation elongation factor [TEF1], fungal DNA damage-responsive protein [DDRP], fungal [e.g., Fusarium or Neurospora crassa] hexagonal peroxisome [Hex1], fungal [e.g., Neurospora crassa] catalase), and any other protein produced at high level in the recombinant fungal host cell.

Non-limiting examples of suitable promoters for use in recombinant bacterial or yeast host cells include promoters, and functional parts thereof, of genes encoding any of the following proteins: LAC4, T7 polymerase, TAC, GAL1, λPL, λPR, beta-lactamase, spa, CYC1, TDH3, GPD, TEF1, ENO2, PGL1, GAP, SUC2, ADH1, ADH2, HXT7, PHO5, CLB1, AOX1, cellulase, amylase, protease, xylanase, and any other protein produced at high level in the recombinant bacterial or yeast host cell.

In some embodiments, the promoter is a promoter of a stress (e.g., heat shock) response gene (e.g., hac1, BIP).

The signal sequence may be any suitable signal sequence that is functional in the recombinant host cell. Non-limiting examples of signal sequences for use in recombinant fungal host cells include signal sequences from Trichoderma reesei (e.g., signal sequence of Cbh1, cbh2, eg11, eg12, xyn1, xyn2, bx11, hfb1, or hfb2), signal sequences from Aspergillus niger (e.g., signal sequence of glaA, amyA, amyC, or aamA), signal sequences from yeast (e.g., signal sequence of alpha mating factor [MFa]), and functional parts thereof. Non-limiting examples of signal sequences for use in recombinant yeast host cells include signal sequences from yeast (e.g., signal sequence of alpha mating factor [MFa]), and functional parts thereof.

The termination sequence may be any suitable termination sequence that is functional in the recombinant host cell. Non-limiting examples of suitable termination sequences for use in recombinant fungal host cells include but are not limited to termination sequences of Aspergillus oryzae (e.g., termination sequence of TAKA amylase gene), Aspergillus niger (e.g., termination sequence of glaA, gpdA, aamA, trpC, pdc1, adh1, amdS, or tef1 gene), Fusarium oxysporum (e.g., termination sequence of serine protease [trypsin] gene), Trichoderma reesei (e.g., termination sequence of cbh1, pdc1, TEF1, gpd1, xyn1, or adh1 gene), Pichia pastoris (e.g., termination sequence of aox1, gap1, adh1, tef1, tps1, or pgk1 gene), Saccharomyces cerevisiae (e.g., termination sequence of adh1, cyc1, gal1, tef1, pdc1, pgk1, or tps1 gene), synthetic termination sequences, and any combination of the above listed sequences. Non-limiting examples of suitable termination sequences for use in recombinant yeast host cells include but are not limited to the PGK1 and TPS1 termination sequences.

The recombinant polynucleotide can further include additional elements. Non-limiting examples of such additional elements include enhancer sequences, response elements, protein recognition sites, inducible elements, protein binding sequences, 5′ and 3′ untranslated regions, polyadenylation sequences, introns, origins of replication, operators (i.e., polynucleotide adjacent to a promoter that comprise a protein-binding domain where a repressor protein can bind and reduce or eliminate activity of the promoter), and selection markers (i.e., genes that encode proteins that can complement the host cell's auxotrophy, provide antibiotic resistance, or result in a color change). Such elements are known in the art. Non-limiting examples of origins of replication include AMA1 and ANSI. Non-limiting examples of suitable selection markers include amdS (acetamidase), argB (ornithine carbamoyltransferase), bar (phosphinothricin acetyltransferase), hph (hygromycin phosphotransferase), niaD (nitrate reductase), pyrG (orotidine 5′-phosphate decarboxylase), sC (sulfate adenyltransferase), and trpC (anthranilate synthase), and derivatives thereof. In some embodiments, the selection marker comprises an alteration that decreases production of the selective marker, thus increasing the number of copies needed to permit a host cell comprising the polynucleotide to survive under selection.

In embodiments in which the recombinant polynucleotide comprises two or more expression cassettes, the operably linked promoters, optional signal sequences, sequences encoding a polypeptide termination sequences, and optional additional elements can be identical or different between the two or more expression cassettes.

Methods for introducing a recombinant polynucleotide into a host cell to obtain a recombinant host cell are well-known in the art. Non-limiting examples of such methods include calcium phosphate transfection, dendrimer transfection, liposome transfection (e.g., cationic liposome transfection), cationic polymer transfection, cell squeezing, sonoporation, optical transfection, protoplast fusion, impalefection, hyrodynamic delivery, gene gun, magnetofection, viral transduction, electroporation and chemical transformation (e.g., using PEG).

In some embodiments, the recombinant polynucleotide is maintained extra-chromosomal in the recombinant host cell on an expression vector (i.e., a polynucleotide that transduces, transforms, or infects a host cell, and causes it to express polynucleotides and/or polypeptides other than those native to the host cell, or in a manner not native to the host cell). In other embodiments, the recombinant polynucleotide is stably integrated within the genome (e.g., a chromosome) of the recombinant host cell. For integration into the genome, the recombinant polynucleotide can comprise sequences for integration into the genome by homologous or nonhomologous recombination. In some embodiments, such sequences enable integration into the host genome at a precise location. The recombinant polynucleotide may comprise at least 100, at least 250, at least 500, at least 750, at least 1,000, or at least 10,000 base pairs that are highly homologous with a target sequence in the genome of the recombinant host cell to enhance the probability of homologous recombination. Such highly homologous sequence may be non-coding or coding. More than one copy of the recombinant polynucleotide may be inserted into the recombinant host cell to increase production of the recombinant milk protein monomer.

The host cell can be a fungal cell, bacterial cell, or protozoa cell. In some embodiments, the host cell is a generally recognized as safe (GRAS) industrial stain.

Examples of suitable fungal cells include but are not limited to filamentous fungal cells, Aspergillus niger, Aspergillus niger var. awamori, Aspergillus oryzae, Candida guilliermondii, Candida lipolytica, Candida pseudotropicalis, Candida utilis, Endothia parasitica, Eremothecium ashbyii, Fusarium moniliforme, Kluyveromyces lactis, Kluyveromyces marxianus var. lactis, Morteirella vinaceae var. raffinoseutilizer, Mucor miehei, Mucor miehei var. Cooney et Emerson, Mucor pusillus Lindt, Myceliophthora thermophile, Penicillium roquefortii, Pichia pastoris, Rhizopus niveus, Saccharomyces cervisea, Saccharomyces fragilis, Trichoderma reesei, and Myceliopthora thermophila.

Examples of suitable bacterial cells include but are not limited to Acetobacter suboxydans, Acetobacter xylinum, Actinoplane missouriensis, Bacillus cereus, Bacillus coagulans, Bacillus licheniformis, Bacillus stearothermophilus, Bacillus subtilis, Escherichia coli, Lactobacillus bulgaricus, Lactococcus lactis, Lactococcus lactis Lancefield Group N, Leuconostoc citrovorum, Leuconostoc dextranicum, Leuconostoc mesenteroides strain NRRL B-512(F), Micrococcus lysodeikticus, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, and Xanthomonas campestris.

Examples of suitable protozoa cells include but are not limited to Tetrahymena thermophile, Tetrahymena hegewischi, Tetrahymena hyperangularis, Tetrahymena malaccensis, Tetrahymena pigmentosa, Tetrahymena pyriformis, and Tetrahymena vorax.

The recombinant host cell can further comprise a genetic modification that improves production of the recombinant milk protein monomer. Non-limiting examples of such genetic modifications include altered promoters, altered kinase activities, altered phosphatase activities, altered protein folding activities, altered protein secretion activities, and altered gene expression induction pathways.

The recombinant host cell can further have reduced or essentially eliminated activity of a protease so as to minimize degradation of the recombinant milk protein monomer (see, for example, PCT application WO 96/29391). Recombinant host cells with reduced or essentially eliminated activity of a protease can be obtained by screening of mutants or by specific genetic modification as per methods known in the art.

The recombinant host cell can further comprise a native or recombinant glycosyltransferase. Non-limiting examples of such endogenous or recombinant glycosyltransferases include fucosyltransferases, galactosyltransferases, glucosyltransferases, xylosyltransferases, acetylases, glucoronyltransferases, glucoronylepimerases, sialyltransferases, mannosyltransferases, sulfotransferases, β-acetylgalactosaminyltransferases, and N-acetylglucosaminyltransferases.

The recombinant host cell can further comprise a native or recombinant kinase or phosphatase. Non-limiting examples of such native or recombinant kinases or phosphatases include protein kinase A, protein kinase B, protein kinase C, creatine kinase B, protein kinase C beta, protein kinase G, TmkA, Fam20 kinases (e.g., Fam20° C.), ATM, CaM-II, cdc2, cdk5, CK1, CKII, DNAPK, EGFR, GSK3, INSR, p38MAPK, RSK, SRC, phosphotransferases, alkaline phosphatase (e.g., UniProtKB—O77578), acid phosphatase, and others (see, for example, Kabir & Kazi. 2011. Genet Mol Biol. 34(4):587).

A recombinant host cell can be cultured in any suitable fermentation vessel known in the art (e.g., culture plate, shake flask, fermentor [e.g., stirred tank fermentor, airlift fermentor, bubble column fermentor, fixed bed bioreactor, laboratory fermentor, industrial fermentor, or any combination thereof]) and at any scale (e.g., small-scale, large-scale) and process (e.g., continuous, batch, fed-batch, or solid state) known in the art.

Suitable culture media include any culture medium in which the recombinant host cell can grow and/or remain viable. In some embodiments, the culture medium is an aqueous medium that comprises a carbon, a nitrogen (e.g., anhydrous ammonia, ammonium sulfate, ammonium nitrate, diammonium phosphate, monoammonium phosphate, ammonium polyphosphate, sodium nitrate, urea, peptone, protein hydrolysates, yeast extract), and a phosphate source. The culture medium can further comprise an inorganic salt, a mineral, a metal, a transition metal, a vitamin, an emulsifying oil, a surfactant, and any other nutrient. Non-limiting examples of suitable carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof. Suitable culture media are available from commercial suppliers or may be prepared according to published compositions (e.g., in catalogues of the American Type Culture Collection).

Suitable conditions for production of the recombinant milk protein monomer are those under which the recombinant host cell can grow and/or remain viable. Non-limiting examples of such conditions include a suitable pH, a suitable temperature, a suitable feed rate, a suitable pressure, a suitable nutrient content (e.g., a suitable carbon content, a suitable nitrogen content, a suitable phosphorus content), a suitable supplement content, a suitable trace metal content, and a suitable level of oxygenation.

In some embodiments, the culture medium further comprises a protease (e.g., a plant-based protease) that can prevent degradation of a recombinant milk protein monomer, a protease inhibitor that reduces the activity of a protease that can degrade the recombinant milk protein monomer, and/or a sacrificial protein that can siphon away protease activity. In some embodiments, the culture medium further comprises a non-natural amino acid for incorporation in the recombinant milk protein monomer produced.

Alternatively, a recombinant milk protein monomer can be obtained using in vitro methods (e.g., using cell-free transcription and/or translation systems).

A native milk protein monomer can be obtained from milk.

Methods for purifying a native or recombinant milk protein monomer from milk or a fermentation broth, respectively, are well-known in the art (see, for example, Protein Purification, J C Janson and L Ryden, Eds., VCH Publishers, New York, 1989; Protein Purification Methods: A Practical Approach, E L V Harris and S Angel, Eds., IRL Press, Oxford, England, 1989). A milk protein monomer can be purified on the basis of its molecular weight, for example, by size exclusion/exchange chromatography, ultrafiltration through membranes, gel permeation chromatography (e.g., preparative disc-gel electrophoresis), or density centrifugation. A milk protein monomer also can be purified on the basis of its surface charge or hydrophobicity/hydrophilicity, for example, by isoelectric precipitation, anion/cation exchange chromatography, isoelectric focusing (IEF), or reverse phase chromatography. A milk protein monomer also can be purified on the basis of its solubility, for example, by ammonium sulfate precipitation, isoelectric precipitation, surfactants, detergents, or solvent extraction. A milk protein monomer also can be purified on the basis of its affinity to another molecule, for example, by affinity chromatography, reactive dyes, or hydroxyapatite. Affinity chromatography can include the use of an antibody having a specific binding affinity for the milk protein monomer, or nickel NTA for a His-tagged recombinant milk protein monomer, or a lectin to bind to a sugar moiety on a recombinant milk protein monomer, or any other molecule that specifically binds the milk protein monomer. In some embodiments, the recombinant milk protein monomer carries a tag that facilitates purification. Non-limiting examples of such tags include epitope tags and protein tags. Non-limiting examples of epitope tags include c-myc, hemagglutinin (HA), polyhistidine (6x-HIS), GLU-GLU, and DYKDDDDK (FLAG) epitope tags. Non-limiting examples of protein tags include glutathione-S-transferase (GST), green fluorescent protein (GFP), and maltose binding protein (MBP). An epitope or protein tag may be removed following isolation of the recombinant milk protein monomer (e.g., via protease cleavage). In embodiments in which a recombinant milk protein monomer is secreted by a recombinant host cell, the recombinant milk protein monomer can be purified directly from the culture medium. In other embodiments, a recombinant milk protein monomer can be purified from a cell lys ate.

In some embodiments, the recombinant milk protein monomer is purified to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth or preparation. In some embodiments, the recombinant milk protein monomer is purified to be at least 2-fold, at least 3-fold, at least 4-fold, at least 5-fold, at least 6-fold, at least 7-fold, at least 8-fold, at least 9-fold, or at least 10-fold more abundant relative to other components in the preparation than it was present in the fermentation broth. In some embodiments, the recombinant milk protein monomer is purified to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by weight.

In some embodiments, the milk protein monomer preparation is spray dried or concentrated via evaporation. In some such embodiments, the preparation comprises a moisture content of less than 10%, less than 7%, less than 5%, less than 3%, or less than 1%.

The identity of the recombinant milk protein monomer can be confirmed and/or quantified by high performance liquid chromatography (HPLC), Western blot analysis, Eastern blot analysis, polyacrylamide gel electrophoresis, capillary electrophoresis, formation of an enzyme product, disappearance of an enzyme substrate, and 2-dimensional mass spectroscopy (2D-MS/MS) sequence identification.

Post-processing of a native or recombinant milk protein monomer can comprise fragmenting (e.g., by chemical means or by exposure to protease enzymes [e.g., trypsin, pepsin, calmapepsin]), removing reactive sites (e.g., removing reactive sites of methionine and/or tryptophan residues by oxidation), modulating (e.g., via chemical, photochemical, and/or enzymatic strategies), cyclizing, biotinylating (i.e., attaching biotin), and conjugation to other elements (e.g., poly-ethylene-glycol, antibodies, liposomes, phospholipids, DNA, RNA, nucleic acids, sugars, disaccharides, polysacharides, starches, cellulose, detergents, cell walls).

Post-processing can occur in a random manner or in a site-specific manner (e.g., at sufhydryl groups of cystein residues [e.g., for aminoethylation, formation of iodoacetamides, formation of maleimides, formation of Dha, covalent attachment via disulfide bonds, and desulfurization]; primary amine groups of lysine residues [e.g., for attachment of activated esters, sulfonyl chlorides, isothiocyanates, unsaturated aldehyde esters, and aldehydes]; phenolic hydroxyl groups of tyrosine residues; removal of specific epitopes [e.g., glycan groups] that can elicit immune responses in humans; azo-electrocyclization).

Post-processing may alter certain chemical and/or physical properties of the native or recombinant milk protein monomer, including but not limited to size, charge, hydrophobicity, hydrophilicity, solvation, protein folding, and chemical reactivity.

### Polymerizing Milk Protein Monomers

Methods for polymerizing protein monomers are known in the art, and can be used in the methods provided herein for polymerizing the milk protein monomers. Non-limiting examples of such methods include methods that employ crosslinking agents, crosslinking enzymes, oxidation (e.g., using oxidizing agents), reduction (e.g., using reducing agents), radiation (e.g., using UV, gamma, electron beam), mechanical agitation (e.g., spinning), pressure and/or heating (e.g., extrusion), turbulence, friction, pH changes, photo-oxidative treatment (e.g., using photo-reactive amino acid analogs), 3D-printing, and combinations thereof.

Non-limiting examples of crosslinking agents include activating agents (i.e., chemicals that activate functional groups on proteins and thus connect proteins without incorporating a spacer), monofunctional crosslinkers (i.e., chemicals with one reactive group that target proteins at one functional group), bifunctional crosslinkers (i.e., chemicals with two reactive groups that target proteins at two functional groups; e.g., homobifunctional crosslinkers (i.e., chemicals with two reactive groups that targets proteins at two identical functional groups), heterobifunctional crosslinkers (chemicals with two reactive groups that targets proteins at two different functional groups), and multifunctional crosslinkers (i.e., chemicals with more than two reactive groups that target proteins at more than two functional groups). Non-limiting examples of crosslinking agents include carbodiimides (e.g., EDC), 2-benzothiazolethiol, and tetramethylthiuram disulfide, formaldehyde, glutaraldehyde, glyceraldehyde, imidoesters (e.g., dimethyl pimelimidate (DMP), dimethyl adipimidate (DMA), dimethyl suberimidate (DMS), N-hydroxysuccinimide (NHS) esters (e.g., dithiobissuccinimidylpropionate, NHS-bromoacetate, BS3), genipin, thioesters, 2,2-dimethoxy-2-phenyl acetophenone, sodium cyanoborohydride, polyglutaraldehyde, pyridinyldisulfide, nitrophenylazide, maleimide reactive groups, N-succinimidyl, iodoacetate, carbonyldiimidazole, dicyclohexylcarbodiimide, SMCC, Sulfo-SMCC, gossypol, tannic acid, lactic acid, formol, glyoxal, vinylsilane, diethyl squarate, oxalic acid, functionalized biopolymers (e.g., poly(ethylene glycol) (PEG) diacrylate, oxidized b-cyclodextrin, telechelic-PVA, dialdehydes derived from PEG or scleroglucan), methylene bis-acrylamide, and bifunctional monosaccharides of variable carbon chain lengths (n=2, 4, 6; e.g., N,N-suberoyl glucosamine, N,N-hexamethylene glucuronamide, or bis-1,1 -(1,8-oc tyl)-glucofuranos idurono-6,3-lactone types). In some embodiments, crosslinking leads to the incorporation of molecular spacer groups between the milk protein monomers. In some embodiments, crosslinking leads to the incorporation of molecular spacer groups between the milk protein monomers and optional non-milk protein monomers.

Non-limiting examples of crosslinking enzymes include transferases (EC 2; e.g., transglutaminases that catalyze a trans-amidation reaction between glutamyl and lysyl side chains of proteins (EC 2.3.2.13; e.g., fibrin-stabilizing factor XIII, microbial transglutaminases, bacterial transglutaminases), hydrolases (EC 3; e.g., sortase SrtA (EC 3.4.22.70), disulfide isomerase (EC 5.3.4.1), oxidoreductases (EC 1; e.g., tyrosinases (EC 1.14.18.1), laccases (EC 1.10.3.2), peroxidases (EC 1.11.1.x), lysyl oxidases (EC 1.4.3.13), and sulfhydryl (or thio) oxidases (EC 1.8.3.2). In some embodiments, the enzymes are used in preparations suitable for applications in the food industry (i.e., are tested for toxicity and immunogenicity, and are certified with the status Generally Recognized as Safe (GRAS) defined by the US Food and Drug Association (FDA). In some such embodiments, the enzymes are GRAS-certified transglutaminase preparations from S. mobaraensis.

In some embodiments, polymerizing of milk protein monomers (e.g., β-lactoglobulin monomers) is achieved at reduced temperature (e.g., less than 30° C., less than 25° C., less than 20, less than 15° C., less than 10, less than 5° C., less than 0° C., less than −5° C., or less than −10° C.; or between 30° C. and −15° C., −10° C., −5° C., 0° C., 5° C., 10° C., 15° C., 20° C., or 25° C.; between 25° C. and −15° C., −10° C., −5° C., 0° C., 5° C., 10° C., 15° C., or 20° C.; between 20° C. and −15° C., −10° C., −5° C., 0° C., 5° C., 10° C., or 15° C.; between 15° C. and −15° C., −10° C., −5° C., 0° C., 5° C., or 10° C.; between 10° C. and −15° C., −10° C., −5° C., 0° C., or 5° C.; between 5° C. and −15° C., −10° C., −5° C., or 0° C.; between 0° C. and −15° C., −10° C., or −5° C.; between −5° C. and −15° C., or −10° C.; or between −10° C. and −15° C.). In some embodiments, polymerizing of milk protein monomers is achieved at elevated temperature (e.g., greater than 30° C., greater than 35° C., greater than 40° C., greater than 45° C., greater than 50° C., greater than 55° C., greater than 60° C., greater than 65° C., or greater than 70° C.; or between 30° C. and 80° C., 75° C., 70° C., 65° C., 60° C., 55° C., 50° C., 45° C., 40° C., or 35° C.; between 35° C. and 80° C., 75° C., 70° C., 65° C., 60° C., 55° C., 50° C., 45° C., or 40° C.; between 40° C. and 80° C., 75° C., 70° C., 65° C., 60° C., 55° C., 50° C., or 45° C.; between 45° C. and 80° C., 75° C., 70° C., 65° C., 60° C., 55° C., or 50° C.; between 50° C. and 80° C., 75° C., 70° C., 65° C., 60° C., or 55° C.; between 55° C. and 80° C., 75° C., 70° C., 65° C., or 60° C.; between 60° C. and 80° C., 75° C., 70° C., or 65° C.; between 65° C. and 80° C., 75° C., or 70° C.; between 70° C. and 80° C., or 75° C.; or between 75° C. and 80° C.).

In some embodiments, polymerizing of milk protein monomers (e.g., β-lactoglobulin monomers) is achieved at acidic pH (e.g., less than 7, less than 6.5, less than 6, less than 5.5, less than 5, less than 4.5, less than 4, less than 3.5, less than 3, less than 2.5, less than 2, or less than 1.5; or between 1.5 and 7, 6.5, 6, 5.5, 5, 4.5, 4, 3.5, 3, 2.5, or 2; between 2 and 7, 6.5, 6, 5.5, 5, 4.5, 4, 3.5, 3, or 2.5; between 2.5 and 7, 6.5, 6, 5.5, 5, 4.5, 4, 3.5, or 3; between 3 and 7, 6.5, 6, 5.5, 5, 4.5, 4, or 3.5; between 3.5 and 7, 6.5, 6, 5.5, 5, 4.5, or 4; between 4 and 7, 6.5, 6, 5.5, 5, or 4.5; between 4.5 and 7, 6.5, 6, 5.5, or 5; between 5 and 7, 6.5, 6, or 5.5; between 5.5 and 7, 6.5, or 6; between 6 and 7, or 6.5; or between 6.5 and 7).

In some embodiments, polymerizing of milk protein monomers (e.g., β-lactoglobulin monomers) is achieved at basic pH (e.g., greater than 7, greater than 7.5, greater than 8, greater than 8.5, greater than 9, greater than 9.5, greater than 10, greater than 10.5, greater than 11, greater than 11.5, greater than 12, greater than 12.5, or greater than 13; or between 7 and 14, 13.5, 13, 12.5, 12, 11.5, 11, 10.5, 10, 9.5, 9, 8.5, 8, or 7.5; between 7.5 and 14, 13.5, 13, 12.5, 12, 11.5, 11, 10.5, 10, 9.5, 9, 8.5, or 8; between 8 and 14, 13.5, 13, 12.5, 12, 11.5, 11, 10.5, 10, 9.5, 9, or 8.5; between 8.5 and 14, 13.5, 13, 12.5, 12, 11.5, 11, 10.5, 10, 9.5, or 9; between 9 and 14, 13.5, 13, 12.5, 12, 11.5, 11, 10.5, 10, or 9.5; between 9.5 and 14, 13.5, 13, 12.5, 12, 11.5, 11, 10.5, or 10; between 10 and 14, 13.5, 13, 12.5, 12, 11.5, 11, or 10.5; between 10.5 and 14, 13.5, 13, 12.5, 12, 11.5, or 11; between 11 and 14, 13.5, 13, 12.5, 12, or 11.5; between 11.5 and 14, 13.5, 13, 12.5, or 12; between 12 and 14, 13.5, 13, or 12.5; between 12.5 and 14, 13.5, or 13; between 13 and 14, or 13.5; or between 13.5 and 14).

In some embodiments, polymerizing of milk protein monomers (e.g., β-lactoglobulin monomers) is achieved at acidic pH and elevated temperature.

In some embodiments, the methods further comprise the step of rupturing intramolecular and/or intermolecular bonds within the milk protein monomers prior to polymerizing the milk protein monomers. In some such embodiments, the intramolecular and/or intermolecular bonds are ruptured using oxidizing and/or reducing agents.

In some embodiments, the methods further comprise the step of adding, at any step during the polymerizing, one or more non-milk protein monomers disclosed herein. In some such embodiments, the non-milk protein monomers are obtained from natural sources (e.g., plants, microbes). Methods for isolating proteins from natural sources are known in the art. Suitable methods include but are not limited to methods that provide protein isolates, protein concentrates, protein flours, and partially purified or purified proteins from natural sources. In other embodiments, the non-milk protein monomers are produced recombinantly (e.g., using similar methods as disclosed herein for recombinantly producing the milk protein monomers).

Polymer formation can be monitored by methods known in the art. Non-limiting examples of such methods include swelling experiments.

### Additional Steps

The method can further comprise the step of adding, at any step during the preparation of the polymer, a native or recombinant non-milk protein monomer disclosed herein.

The native non-milk protein monomers can be obtained from natural sources (e.g., milk, plants, microbes). Methods for isolating proteins from natural sources are known in the art. Suitable methods include but are not limited to methods that provide protein isolates, protein concentrates, protein flours, and partially purified or purified proteins from natural sources. The recombinant non-milk protein monomer can be produced by methods known in the art (e.g., by methods similar to those disclosed herein for production of the recombinant milk protein monomers provided herein using recombinant host cells that comprise recombinant polynucleotides encoding such non-milk protein monomers). The native or recombinant non-milk protein monomer can be non-purified protein, partially purified protein, or purified protein. The native or recombinant non-milk protein monomer can be hydrolyzed prior to use. Hydrolyzing can be accomplished chemically or enzymatically (e.g., using proteases such as trypsin, pepsin, or chymotrypsin).

### Post-Processing of Polymers

Post-processing of a polymer can involve, for example, isolating (i.e., purifying; e.g., via ultracentrifigation), dehydrating (e.g., via spray drying, roller drying, fluid bed drying, freeze drying, drying with ethanol, evaporating), and coating (e.g., with other polymers, heparin, pectin, alginate). Methods for post-processing polymers are known in the art.

### Composition Comprising Polymer of Milk Protein Monomers

In another aspect, provided herein is a composition that comprises a polymer provided herein.

In some embodiments, the composition comprises at least 0.001%, 0.01%, 0.1%, 1%, 10%, 20%, 30%, 40%, 50%, 60%, 70%, 80%, 90%, 95%, or 99%; between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100% by weight of a polymer provided herein.

In some embodiments, the composition does not comprise at least one component found in a similar composition comprising a petroleum-based polymer; and/or comprises a higher or lower concentration of at least one component found in a similar composition comprising a petroleum-based polymer.

In some embodiments, the composition does not comprise at least one component found in fresh milk (e.g., fresh bovine milk, fresh goat milk, fresh sheep milk); and/or comprises a higher or lower concentration of at least one component found in fresh milk. Non-limiting examples of such components include lactose and all native milk proteins (e.g., immunoglobulins, lactoferrin).

In some embodiments, the composition comprises a polymeric network. In some embodiments, a polymeric network comprises covalent crosslinks, including but not limited to amide bonds (e.g., lactam bridges, native chemical ligation bonds, Staudinger ligation bonds) and disulfide bonds. In some embodiments, the average densities of the crosslinks of a polymeric network are 1, 2, 3, 4, 5, 6, 7, 8, 9, 10, more than 10, more than 12, more than 14, more than 16, more than 18, or more than 20 crosslinks per subunit comprised in the polymers. In some embodiments, the crosslinks are cleavable (e.g., by treatment with thiols (e.g., β-mercaptoethanol, dithiothreitol). In some embodiments, the polymeric network comprise a single polymer provided herein, or two or more different polymers provided herein (i.e., the polymeric network is a “hybrid polymer”).

In some embodiments, the composition is an adhesive (i.e., a material that forms an adhesive bond; e.g., glue, wallpaper adhesive, wood adhesive, paper adhesive, cork adhesive, chipboard adhesive, surgical/medical glue, cement, mucilage, paste). In some such embodiments, the adhesive comprises at least 80% (e.g., at least 80%, at least 85%, at least 90%, at least 95%) by weight of a recombinant milk protein monomer (e.g., one or more recombinant whey protein monomers (e.g., only recombinant β-lactoglobulin, only recombinant α-lactalbumin, recombinant β-lactoglobulin and recombinant α-lactalbumin), one or more recombinant casein monomers (e.g., only recombinant κ-casein monomer, only recombinant β-casein monomer, recombinant κ-casein monomer and/or recombinant β-casein and/or γ-casein monomer), or mixtures of at least one recombinant whey protein monomer and at least one recombinant casein monomer (e.g., recombinant β-lactoglobulin and recombinant κ-casein, recombinant α-lactalbumin and recombinant κ-casein, recombinant β-lactoglobulin and recombinant α-lactalbumin and recombinant κ-casein).

In some embodiments, the composition is a hard or medium-hard plastic (e.g., bottle, button, window, pen, fiber [e.g., yarn, textile, carpet, curtain, clothing). In some embodiments, the composition is a soft plastic (e.g., bag, wrap, edible film, waterproof film, contact lens, packaging material). In some such embodiments, the plastic comprises at least 80% (e.g., at least 80%, at least 85%, at least 90%, at least 95%) by weight of a recombinant milk protein monomer (e.g., one or more recombinant whey protein monomers (e.g., only recombinant β-lactoglobulin, only recombinant α-lactalbumin, recombinant β-lactoglobulin and recombinant α-lactalbumin), one or more recombinant casein monomers (e.g., only recombinant κ-casein monomer, only recombinant β-casein monomer, recombinant κ-casein monomer and/or recombinant β-casein and/or γ-casein monomer), or mixtures of at least one recombinant whey protein monomer and at least one recombinant casein monomer (e.g., recombinant β-lactoglobulin and recombinant κ-casein, recombinant α-lactalbumin and recombinant κ-casein, recombinant β-lactoglobulin and recombinant α-lactalbumin and recombinant κ-casein).

In some embodiments, the composition is a coating or facing (e.g., glossy coating, protective coating, varnish, coating for medical tablet, paper coating, painting, leather finishing, textile coating). In some embodiments, the composition is an industrial polymer (i.e., compounds used in the manufacture of synthetic industrial materials). In some embodiments, the composition is a pharmaceutical formulation (e.g., product used for delivery of a medicinal agent (e.g., micro- or nano-particle (e.g., bead, micelle) that encapsulates a therapeutic or nutraceutical for delivery (e.g., controlled delivery), coating of tablet, capsule, compact, hydrogel). In some embodiments, the composition is a medical diagnostic. In some embodiments, the composition is a hemostatic product. In some embodiment, the composition is a gel (e.g., hydrogel for controlled release of a therapeutic, hydrogel for immobilizing a protein (e.g., enzyme). In some embodiments, the composition is a biocompatible material (e.g., implant, self-dissolving suture, bone-replacing composite, material supporting nerve repair, scaffold for growing cells, prosthetic implant, membrane for promoting wound healing, tissue-engineering scaffolding). In some such embodiments, the implant is soluble until a specific condition (e.g., pH, temperature) triggers the formation of a polymeric network. In some embodiments, the composition is a lubricant. In some embodiments, the composition is a piece of furniture. In some embodiments, the composition is a cosmetic or personal care product (e.g., ointment, lotion, cream (e.g., moisturizing cream), cleanser, massage cream, soap, hair shampoo, hair conditioner, skin mask, finishing product, hair tonic). In some embodiments, the composition is a gum. In some embodiments, the composition is a paper (e.g., paper sheet, paper label, packaging paper, photographic support).

In some embodiments, the composition is biodegradable. In some such embodiments, the composition is biodegradable under anaerobic condition. In other such embodiments, the composition is biodegradable under aerobic condition.

In some embodiments, the composition is biocompatible. In some such embodiments, the composition does not comprise an allergenic epitope. In some embodiments, the composition is capable of growth (e.g., comprises non-polymerized milk protein monomers that polymerize over time or under specific conditions (e.g., temperature, oxygenation, pH, pressure, shear).

In some embodiments, the composition comprises polymers provided herein having various densities. In some such embodiments, the composition comprises layers of polymers provided herein having different densities.

In some embodiments, the composition is a biological scaffold (e.g., a mesh structure that mimics a natural extracellular matrix and entraps bioactive molecules such as growth factors and thereby supports 3D tissue formation).

All publications, patents, patent applications, sequences, database entries, and other references mentioned herein are incorporated by reference to the same extent as if each individual publication, patent, patent application, sequence, database entry, or other reference was specifically and individually indicated to be incorporated by reference. In case of conflict, the present specification, including definitions, will control. The terminology and description used herein is for the purpose of describing particular embodiments only and is not intended to limit the invention.

### EXAMPLES

The following examples are included to illustrate specific embodiments of the invention. The techniques disclosed in the examples represent techniques discovered by the inventors to function well in the practice of the invention; however, those of skill in the art should, in light of the present disclosure, appreciate that many changes can be made in the specific embodiments that are disclosed and still obtain a like or similar result without departing from the spirit and scope of the invention. Therefore, all matter set forth or shown in the examples is to be interpreted as illustrative and not in a limiting sense.

### Example 1: Production of Recombinant Milk Protein Monomers in Recombinant Kluyveromyces Lactis Host Cells

Protein sequences of bovine α-S1 casein (UniProt accession #P02662), bovine α-2 casein (UniProt accession #P02663), bovine β-casein (UniProt accession #P02666), bovine κ-casein (UniProt accession #P02668), bovine α-lactalbumin (UniProt accession #B6V3I5) and bovine β-lactoglobulin (UniProt accession #P02754) were obtained on Uniprot.org, and altered with the following changes: removed 15 or 21-residue signal peptide from N-terminal end, added XhoI (CTC GAG) endonuclease recognition sequence and KEX endopeptidase recognition sequence (AAA AGA) to 5′ end of DNA, and added SalI (GTC GAC) endonuclease recognition sequence to 3′ end of DNA. An additional combination sequence was made by combining the sequences for the four caseins in the order shown above, separating each sequence with the following DNA phrase: GGC TCA GGA TCA GGG TCG AAA AGA GGC TCA GGA TCA GGG TCG, wherein the non-underlined segments encode a (GS)₆ linker sequence for adequate posttranslational spacing and accessibility to the KEX protease, and the underlined segment encodes the KEX endopeptidase sequence that cleaves the proteins apart post-translation. As above, the entire cassette is flanked on the 5′ end by XhoI and on the 3′ end by SalI for ligation into pKLAC2 (New England Biolabs, Beverly, Mass.). DNA was synthesized by either Gen9, Inc. (Cambridge, Mass.) or IDT (Coralville, Iowa). The plasmid further comprised a multiple cloning site, a Lac promoter, an acetamide-based reporter gene, and the alpha-mating factor gene as a fusion protein for secretion of exogenous proteins.

Transfection of competent Kluyveromyces lactis host cells (New England Biolabs kit; Catalog #E1000S) was accomplished by thawing a tube of 0.5 mL cells containing 25% glycerol on ice and adding 0.62 mL yeast transfection reagent. The mixture was warmed at 30° C. for 30 minutes, heat shocked at 37° C. for 1 hour. The cells were pelleted at 7,000 rpm, and washed twice with 1.0 mL of YPGal medium. The cell pellet was resuspended in 1 mL sterile 1×PBS. 10 μL, 50 μL, and 100 μL of the cell suspension was placed into separate fresh sterile 1.5 mL microcentrifuge tubes each containing 50 μL of sterile deionized water. Tubes were mixed briefly and spread onto separate yeast carbon base agar (YCB Agar) plates containing 5 mM acetamide for selection. Plates were incubated, inverted, at 30° C. for 4 days until colonies formed. Individual colonies were streaked onto fresh YCB agar plates containing 5 mM acetamide, and incubated at 30° C. for 2 days to create a homozygous culture plate. Each plate was tested for successful integration of the vector plasmid, followed by PCR analysis to test for multiple integrants of the vector. To test for protein expression, a single colony from each plate was added to a 10 mL glass culture tube containing 2 mL YPGal media, and grown for 2 days, before the cells were pelleted and the supernatant was run on an SDS PAGE gel. The strains that provided the best protein expression were scaled up to a 10 mL, 100 mL, 500 mL, and ultimately 1 L culture vessel.

Scale-up cultures are seeded in a 1 L shake flask at split ratios of at least 1:10. Prior to seeding, inoculation flasks are grown for 24 hours in production media without acetamide supplementation. On the starting day of a fed-batch production run, the reactor is charged with 90% of the target starting volume and heated to a 30° C. run temperature. Additional parameters can be explored in the process optimization phase. When the reactor reaches 30° C., the inoculation flask is added to the reaction vessel dropwise using a peristaltic pump. The reactor is maintained using vendor supplied software at a target pH. Twice daily samples are taken of the reactor broth to quantify the amount of glucose and electrolyte usage by the cells, and to confirm the reactor's pH and dissolved gas measurements. After each measurement, bolus glucose is added to maintain a target glucose concentration 10% (this may also be altered in process development). When cells reach maximum density, protein production is triggered by the addition of galactose. Optimum run length can be determined in process development as well, but is set as a 5-day fed-batch culture. After a full run, the recombinant Kluyveromyces lactis host cells are removed from the reactor and the recombinant milk protein is purified.

The recombinant milk protein can be isolated using methods known in the art. For example, α-s1casein, α-s2casein, and β-casein are inherently hydrophobic, and precipitate out when they are secreted from the recombinant Kluyveromyces lactis host cell and come into contact with water. Purification can also occur based on molecular weight (e.g., using membrane filtration [e.g., ultrafiltration, microfiltration] using membranes with suitable pore sizes) and/or isoelectric point (IEP) precipitation (the IEPs of native milk proteins are as follows: α-lactalbumin 4.2, β-lactoglobulin 5.2, α-casein 4.1, β-casein 4.5, κ-casein 4.1, and γ-casein 5.8-6.0; at a pH near the IEP of a protein, the charges on the protein are neutralized, causing the protein to fall out of solution).

For protein purification, the 2 L of culture was spun at 3,000 g in a floor centrifuge to pellet out the recombinant Kluyveromyces lactis host cells. The pellet was discarded, and the supernatant was transferred into a new vessel. The pH of the solution was lowered to the IEP of the recombinant milk protein to be isolated (using, for example, lactic, acid hydrochloric, acid or sulphuric acid). This was followed by incubation of the supernatant at 35° C. for 30 minutes in a shaker flask, and centrifugation at 14,000 g in a floor centrifuge to pellet out the protein mixture. The solid pellet and the supernatant solution were run on a 14% SDA-PAGE gel to check for protein expression. Further characterization can be done to confirm primary amino acid sequence, glycosylation, and phosphorylation of the isolated protein.

The isolated protein can be heated to expel the moisture, and/or spray dried to obtain a milk protein monomer powder.

### Example 2: Production of Recombinant Milk Protein Monomers in Recombinant Pichia Pastoris Host Cells

A recombinant Pichia pastoris host cell was generated by transforming Pichia pastoris (Komagataella phaffii) strain BG12 (Biogrammatics, Carlsbad, Calif.) with a vector comprising a Bos taurus milk protein (e.g., β-lactoglobulin) coding sequence that was codon-optimized for expression in yeast and fused in frame to an N-terminal secretion signal (pre or pre-pro signal peptide region of the alpha mating factor of Saccharomyces cerevisiae, native milk protein signal peptide, OST1 signal peptide). The milk protein coding sequence was flanked by a promoter (constitutive promoter pGAP or pPGK, or methanol-inducible promoter pAOX1) and a terminator (tAOX1 pA signal). The vector further comprised dominant resistance markers for selection of bacterial and yeast transformants, and a bacterial origin of replication. The vector was transformed into the Pichia pastoris host cells using a heat-shock protocol to generate recombinant host strains comprising an integrated copy of the milk protein coding sequence. Transformants were plated on minimal media and incubated for 48 hours at 30° C. Clones from each final transformation were inoculated into 300 μL of Buffered Minimal Media in 96-well blocks, and incubated for 7 two or more than 100 hours at 30° C. with agitation at 600 rpm. A sample was removed, the recombinant host cells were pelleted via centrifugation, and the fermentation supernatant was recovered. ELISA assays and SDS-PAGE gel analyses were performed with the supernatant samples to identify positive transformants.

The recombinant Pichia pastoris host cell was grown in a minimal basal media containing phosphate and nitrogen salts with 80% of glycerol as a starting feedstock, in a stirred fermentation vessel controlled at 30□, 1 V/Vm of air flow, and minimum agitation of 100 rpm. The pH of the fermentation broth was controlled at 5.0 with on-demand addition of ammonium hydroxide. Once batch glycerol was depleted, glycerol was added via a programmed feed recipe that delivered glycerol at a rate of 6 g/L/h. The oxygen demand of the strain was satisfied by controlling agitation rate as demanded. When agitation was no longer able to maintain the dissolved oxygen set-point, 100% oxygen gas was sparged into the vessel to control dissolved oxygen. The pH of fermentation was shifted from 5.0 to 3.0 once batched glycerol had been depleted. Antifoam C was added as needed to control foam. The fermentation was harvested after at least 100 hours, at about cell density of 600-800 at OD₆₀₀. Biomass was removed from the broth by centrifugation at 5,000 g. The supernatant was concentrated over 100 kDa MWCO membranes. The concentrate retentate was diafiltered over 5 kDa MWCO membranes into 50 mM imidazole, pH 6.8. The concentrated retentate was passed over a Q Sepharose® Fast Flow column. The mobile phase was 50 mM imidazole, pH 6.8, and the recombinant β-lactoglobulin was eluted on a 2M NaCl gradient. The gradient was run from 0-30% over 30 column volumes. Peak fractions were collected and analyzed on RP-HPLC. Peaks containing recombinant β-lactoglobulin with a purity of >85% were pooled for final diafiltration into water.

### Example 3: Production of Recombinant β-Lactoglobulin Monomers in Recombinant Trichoderma Reesei Host Cells

A recombinant Trichoderma reesei host cell was generated by transforming Trichoderma reesei (Hypocrea jecorina) strain Qm6a with a vector comprising a Bos taurus β-lactoglobulin coding sequence. ELISA assays and SDS-PAGE gel analyses were performed with the supernatant samples to identify positive transformants.

The recombinant Trichoderma reesei host cell was grown in a minimal basal media containing inorganic salts as sources of phosphate, ammonium, magnesium, potassium, sodium, sulfate, chloride, calcium, iron, manganese, zinc, molybdenum, copper, cobalt, and borate, with a carbohydrate-based carbon source as a starting feedstock, in a stirred fermentation vessel controlled at temperatures ranging between 25□ through 34□, at aeration rates between 0.2 V/Vm and 1 V/Vm, and a minimum agitation to ensure proper mixing and dispersion of biomass and nutrients including oxygen (as delivered in compressed air). The pH of the fermentation broth was controlled at various set points ranging from 3.0 to 5.5 with on-demand addition of ammonium hydroxide. Once the batch carbohydrate was depleted, a solution containing glucose or lactose was added via a programmed feed recipe that delivered the carbon source at specific feed rates ranging from 0.01 g through 0.1 g dry substrate per gram dry cell mass (“DCM”) per hour. The oxygen demand of the culture was satisfied by controlling agitation rate as demanded to maintain a target dissolved oxygen set point ranging from 5% to 50%. When agitation was no longer able to maintain the target dissolved oxygen set-point, aeration was increased up to 2.0 V/Vm. Once the culture was running at maximum agitation and aeration, the dissolved oxygen was allowed to drop below setpoint with no further actions taken. Various antifoams, including but not limited to ACP 1500, Antifoam 204 (Sigma), Erol DF6000K, Hodag K-60K, Industrol DF204 (BASF), P-2000E (Dow), SAG 471, SAG 5693, SAG 710, SAG 730, Silicone Antifoam (Sigma), Struktol J647, Struktol J673A, and sunflower oilwere added as needed to control foaming in the fermentations. Each fermentation was harvested after at least 120 hours, at biomass concentrations of typically between 20 g and 50 g dry cell weight (“DCW”) per liter. Biomass was removed from the broth by centrifugation at 4,000×g for 20 min, and the recombinant β-lactoglobulin was purified.

### Example 4: Production of Polymer Comprising Recombinant Whey Protein Monomers

The recombinant β-lactoglobulin of any of Examples 1 through 3 was combined with a weak acid (e.g., 5% acetic acid) or base (e.g., 5% sodium bicarbonate, sodium hydroxide, potassium hydroxide, calcium hydroxide) alone or in combination with α-lactalbumin and/or κ-casein and/or secreted host cell proteins, to a final concentration of between 2% and 10% by weight of the recombinant β-lactoglobulin, a final pH of between 2 and 8, and a final conductivity of between 10 ms/cm and 300 mS/cm. The mixture was heated at at least 79 C for 22 minutes. The solid polymer aggregate was captured by centrifugation (e.g., at 4,000 g for 20 min), filtration, solvent extractions, chromatography, or other method. The solid polymer aggregate was dried to a desired moisture content that still permits shaping. After shaping, the polymer is further dried to set a final form.

### Example 5: Production of Adhesive Comprising Recombinant Casein Monomers

One or more of the recombinant caseins of any of Examples 1 through 4 is dissolved in 50 mL of water, to which is added 5 g of baking soda (sodium bicarbonate; alternatives: hydrated lime, calcium phosphate). Drops of water are added slowly while stirring until a white casein glue is obtained.

### Example 6: Extrusion of Recombinant Milk Protein Monomers and Non-Milk Protein Monomers

Soybean protein isolate (SI) and one or more of the recombinant caseins (CAS) of any of Examples 1 through 4 are compounded with 0%, 10%, or 30% by weight of Al₂O₃ in a co-rotating twin-screw extruder (e.g., Berstorff ZE 25(CL) 40D) in the presence of 10% by weight of glycerol and 30% by weight of water. Further optional ingredients include TCP and/or coupling agent NZ12 (e.g., at 1% by weight). The extruded compounds (in the form of pellets) are molded into ASTM tensile test bars (24 mm\hat{ }2 cross section), after being conditioned (60° C. over 24 hours) until the respective moisture content reaches 10-12%. The specimens are molded using, for example, a DEMAG D25 NC 4, under optimized processing conditions. The injection-molded samples are conditioned at 23° C. and 60% relative humidity (RH) for at least 1 week.

### Example 7: Production of Gel Comprising Recombinant Casein Monomers

One or more of the recombinant caseins of any of Examples 1 through 4 are dissolved at 27.5 g/L in a buffer solution containing 25 mM of 4-(2-hydroxyethyl)-1-piperazine ethanesulfonic acid (HEPES), and 2 mM CaCl2 at pH7 or pH10.5 (higher pH permits arginine site chains to react in addition to lysine side chains). Genipin (cross-linking agent extracted from Gardenia jasminoides) is dissolved in a mixture of 74/26 (w/w) HEPES buffer and absolute ethanol to yield a stock solution of 200 mM. The 2 solutions are combined to obtain a final concentration of 5 mM, 10 mM, or 20 mM genipin at the same final concentration of casein (25 g/L; dilution caused by the addition of genipin is corrected by adding HEPES buffer and/or absolute ethanol). The mixture is incubated at 50° C. for 24 hours to obtain a covalently bound gel matrix.

### Example 8: Production of Film/Sheet Comprising Recombinant Whey Protein Monomers

A solution comprising between 20% and 40% by weight of one or more of the recombinant whey proteins of any of Examples 1 through 4 is heated at 60° C. or higher, and then combined with a synthetic co-polymer (e.g., polyvinyl acetate [PVAC], polyvinyl alcohol [PVA], polyvinyl pyrollidone [PVP]). A chemical crosslinker (e.g., phenol-formaldehyde oligomer, polymeric methylenebisphenyl diisocyanate, glutaraldehyde) or enzymatic crosslinker is added. Alternatively, the whey protein suspension is molded at between 120° C. and 150° C. and 20 MPa pressure for 5 minutes in a hot press, followed by ambient cooling and subsequent annealing overnight in an oven at 50° C., to obtain a polymer of desirable bonding strength.

All publications, patents, patent applications, sequences, database entries, and other references mentioned herein are incorporated by reference to the same extent as if each individual publication, patent, patent application, sequence, database entry, or other reference was specifically and individually indicated to be incorporated by reference. In case of conflict, the present specification, including definitions, will control. The terminology and description used herein is for the purpose of describing particular embodiments only and is not intended to limit the scope of the invention.

What is claimed is:

1. A polymer comprising a milk protein monomer component and having a desirable attribute.

2. The polymer of claim 1 wherein the milk protein monomer component consists of one whey protein monomer.

3. The polymer of claim 2 wherein the one whey protein monomer is a β-lactoglobulin monomer.

4. The polymer of claim 2 wherein the one whey protein monomer is an α-lactalbumin monomer.

5. The polymer of claim 1 wherein the milk protein monomer component consists of one casein monomer.

6. The polymer of claim 5 wherein the one casein monomer is a κ-casein monomer.

7. The polymer of claim 5 wherein the one casein monomer is a β-casein monomer.

8. The polymer of claim 5 wherein the one casein monomer is a γ-casein monomer.

9. The polymer of claim 1 wherein the milk protein monomer component consists of two whey protein monomers.

10. The polymer of claim 9 wherein the two whey protein monomers consist of a β-lactoglobulin monomer and an α-lactalbumin monomer.

11. The polymer of claim 1 wherein the milk protein monomer component consists of two casein monomers.

12. The polymer of claim 11 wherein the two casein monomers consist of a κ-casein monomer and a β-casein monomer.

13. The polymer of claim 11 wherein the two casein monomers consist of a κ-casein monomer and a γ-casein monomer.

14. The polymer of claim 11 wherein the two casein monomers consist of a β-casein monomer and a γ-casein monomer.

15. The polymer of claim 1 wherein the milk protein monomer component consists of a mixture of one whey protein monomer and one casein monomer.

16. The polymer of claim 15 wherein the weight ratio of the whey protein monomer to the casein monomer is between 10 to 1 and 1 to 10.

17. The polymer of claim 15 wherein the mixture of one whey protein monomer and one casein monomer consists of a β-lactoglobulin monomer and a κ-casein monomer.

18. The polymer of claim 15 wherein the mixture of one whey protein monomer and one casein monomer consists of a β-lactoglobulin monomer and a β-casein monomer.

19. The polymer of claim 15 wherein the mixture of one whey protein monomer and one casein monomer consists of a β-lactoglobulin monomer and a γ-casein monomer.

20. The polymer of claim 15 wherein the mixture of one whey protein monomer and one casein monomer consists of an α-lactalbumin monomer and a κ-casein monomer.

21. The polymer of claim 15 wherein the mixture of one whey protein monomer and one casein monomer consists of an α-lactalbumin monomer and a β-casein monomer.

22. The polymer of claim 15 wherein the mixture of one whey protein monomer and one casein monomer consists of an α-lactalbumin monomer and a γ-casein monomer.

23. The polymer of claim 1 wherein the milk protein monomer component consists of two or more whey protein monomers.

24. The polymer of claim 23 wherein the two or more whey protein monomers comprise a β-lactoglobulin monomer and an α-lactalbumin monomer.

25. The polymer of claim 1 wherein the milk protein monomer component consists of two or more casein monomers.

26. The polymer of claim 25 wherein the two or more casein monomers comprise a κ-casein monomer and a β-casein monomer.

27. The polymer of claim 25 wherein the two or more casein monomers comprise a κ-casein monomer and a γ-casein monomer.

28. The polymer of claim 25 wherein the two or more casein monomers comprise a β-casein monomer and a γ-casein monomer.

29. The polymer of claim 25 wherein the two or more casein monomers comprise a κ-casein monomer, a β-casein monomer, and a γ-casein.

30. The polymer of claim 1 wherein the milk protein monomer component consists of a mixture of one or more whey protein monomers and one or more casein monomers.

31. The polymer of claim 30 wherein the weight ratio of total whey protein monomers to total casein monomers is between 10 to 1 and 1 to 10.

32. The polymer of claim 30 wherein the mixture of one or more whey protein monomers and one or more casein monomers comprises a β-lactoglobulin monomer and a κ-casein monomer.

33. The polymer of claim 30 wherein the mixture of one or more whey protein monomers and one or more casein monomers comprises a β-lactoglobulin monomer and a β-casein monomer.

34. The polymer of claim 30 wherein the mixture of one or more whey protein monomers and one or more casein monomers comprises a β-lactoglobulin monomer and a γ-casein monomer.

35. The polymer of claim 30 wherein the mixture of one or more whey protein monomers and one or more casein monomers comprises an α-lactalbumin monomer and a κ-casein monomer.

36. The polymer of claim 30 wherein the mixture of one or more whey protein monomers and one or more casein monomers comprises an α-lactalbumin monomer and a β-casein monomer.

37. The polymer of claim 30 wherein the mixture of one or more whey protein monomers and one or more casein monomers comprises an α-lactalbumin monomer and a γ-casein monomer.

38. The polymer of claim 30 wherein the mixture of one or more whey protein monomers and one or more casein monomers comprises a β-lactoglobulin monomer, an α-lactalbumin monomer, and a κ-casein monomer.

39. The polymer of claim 30 wherein the mixture of one or more whey protein monomers and one or more casein monomers comprises a β-lactoglobulin monomer, an α-lactalbumin monomer, and a β-casein monomer.

40. The polymer of claim 30 wherein the mixture of one or more whey protein monomers and one or more casein monomers comprises a β-lactoglobulin monomer, an α-lactalbumin monomer, and a γ-casein monomer.

41. The polymer of claim 1 wherein the milk protein monomer component comprises a recombinant milk protein monomer.

42. The polymer of claim 41 wherein the recombinant milk protein monomer was produced by a fungal cell or a bacterial cell.

43. The polymer of claim 42 wherein the fungal cell is from a genus selected from the group consisting of Saccharomyces, Kluyveromyces, Pichia, Aspergillus, Tetrahymena, Yarrowia, Hansenula, Blastobotrys, Candida, Zygosaccharomyces, Debrayomyces, and Fusarium.

44. The polymer of claim 41 wherein the recombinant milk protein monomer is a recombinant whey protein monomer.

45. The polymer of claim 44 wherein the recombinant milk protein monomer is a recombinant β-lactoglobulin monomer.

46. The polymer of claim 44 wherein the recombinant milk protein monomer is a recombinant α-lactalbumin monomer.

47. The polymer of claim 41 wherein the recombinant milk protein monomer is a recombinant casein monomer.

48. The polymer of claim 47 wherein the recombinant milk protein monomer is a recombinant κ-casein monomer.

49. The polymer of claim 47 wherein the recombinant milk protein monomer is a recombinant β-casein monomer.

50. The polymer of claim 47 wherein the recombinant milk protein monomer is a recombinant γ-casein monomer.

51. The polymer of claim 41 wherein the recombinant milk protein monomer has a PTM.

52. The polymer of claim 51 wherein the PTM is a non-native PTM.

53. The polymer of claim 52 wherein the non-native PTM is a non-native glycolylation.

54. The polymer of claim 52 wherein the non-native PTM is a non-native phosphorylation.

55. The polymer of claim 41 wherein the recombinant milk protein monomer has a non-native reactive site.

56. The polymer of claim 41 wherein the recombinant milk protein monomer has a milk protein repeat.

57. The polymer of claim 56 wherein the milk protein repeat is consecutive.

58. The polymer of claim 56 wherein the milk protein repeat is non-consecutive.

59. The polymer of claim 1 wherein the milk protein monomer component further comprises a native milk protein monomer.

60. The polymer of claim 59 wherein the native milk protein monomer is a native whey protein monomer.

61. The polymer of claim 59 wherein the native milk protein monomer is a native casein monomer.

62. The polymer of claim 59 wherein the milk protein monomer component comprises a weight ratio of total recombinant milk protein monomers and total native milk protein monomers of between 100 to 1 and 1 to 100.

63. The polymer of claim 59 wherein the milk protein monomer component comprises a weight ratio of total recombinant whey protein monomers and total native whey protein monomers of between 10 to 1 and 1 to 10.

64. The polymer of claim 59 wherein the milk protein monomer component comprises a weight ratio of total recombinant casein monomers and total native casein monomers of between 10 to 1 and 1 to 10.

65. The polymer of claim 1 wherein the polymer further comprises a non-milk protein monomer component.

66. The polymer of claim 65 wherein the polymer comprises a weight ratio of milk protein monomer component to non-milk protein monomer component of between 100 to 1 to 1 to 100.

67. The polymer of claim 65 wherein the non-milk protein monomer component comprises a plant protein monomer.

68. The polymer of claim 67 wherein the plant protein monomer is a pea protein monomer.

69. The polymer of claim 65 wherein the non-milk protein monomer component comprises a fungal protein monomer.

70. The polymer of claim 65 wherein the non-milk protein monomer component comprises a recombinant non-milk protein monomer.

71. The polymer of claim 1 wherein the desirable attribute is selected from the group consisting of a desirable crystallinity, a desirable viscosity, a desirable density, a desirable biodegradability, a desirable adhesiveness, a desirable hardness, a desirable tensile strength, a desirable toughness, a desirable creep or cold flow, a desirable porosity, a desirable electrical conductivity, a desirable thermal conductivity, a desirable elastic modulus, a desirable flexibility, a desirable strength-at-break, a desirable glass transition temperature, and a desirable shaping temperature.

72. The polymer of claim 1 wherein the polymer is essentially free of a component derived from petroleum.

73. A method for producing a polymer, comprising steps for:
obtaining one or more milk protein monomers;
optionally obtaining one or more non-milk protein monomers;
polymerizing the one or more recombinant milk protein monomers (and the optional one or more non-milk protein monomers) under conditions that provide a polymer with a desirable attribute; and
optionally post-processing the polymer.

74. A method of claim 73 wherein at least one of the one or more milk protein monomers is obtained as a recombinant milk protein monomer produced by a recombinant host cell.

75. A method of claim 74 wherein the recombinant host cell is a recombinant fungal cell.

76. A method of claim 75 wherein the fungal cell is selected from a genus selected from the group consisting of Saccharomyces, Kluyveromyces, Pichia, Aspergillus, Tetrahymena, Yarrowia, Hansenula, Blastobotrys, Candida, Zygosaccharomyces, Debrayomyces, and Fusarium.

77. A method of claim 74 wherein the recombinant host cell is a recombinant bacterial cell.

78. A composition comprising the polymer of claim 1.

79. The composition of claim 78 wherein the composition comprises a polymeric network.
